(19) United States
(12) Patent Application Publication
(10) Pub. No.: US 2025/0114004 A1
(43) Pub. Date: Apr. 10, 2025

(54) DEVICE AND METHOD FOR DETECTING ORTHOSTATIC VITAL SIGNS

(71) Applicant: Wayne State University, Detroit, MI (US)

(72) Inventor: Amar S. Basu, Ann Arbor, MI (US)

(21) Appl. No.: 18/910,860

(22) Filed: Oct. 9, 2024

Related U.S. Application Data
(60) Provisional application No. 63/543,168, filed on Oct. 9, 2023.

(51) Int. Cl. A61B 5/0205; A61B 5/00; A61B 5/021; A61B 5/024; A61B 5/11

(52) U.S. Cl. CPC A61B 5/02055; A61B 5/1116; A61B 5/4561; A61B 5/7225; A61B 5/7275; A61B 5/02125; A61B 5/02416

(57) ABSTRACT

A method includes generating photoplethysmogram (PPG) data and motion data continuously. The method also includes in response to detecting, based on the motion data, that an angle associated with a user's posture indicates that the user is in a supine position, associating, by the data processing hardware, the PPG data with a first phase. The method includes in response to detecting, based on the motion data, that the angle associated with the user's posture indicates that the user is in a standing up position or a sitting up position, associating, by the data processing hardware, the PPG data with a third phase. The method further includes determining one or more orthostatic metrics based on the PPG data associated with the first phase and the PPG data associated with the third phase.

### CROSS REFERENCE TO RELATED APPLICATIONS

This U.S. patent application claims priority under 35 U.S.C. § 119(e) to U.S. Provisional Application 63/543,168, filed on Oct. 9, 2023. The disclosure of this prior application is considered part of the disclosure of this application and is hereby incorporated by reference in its entirety.

### TECHNICAL FIELD

This disclosure pertains to systems, devices, and methods for monitoring patient's orthostatic health. More specifically, the disclosure relates to diagnosing orthostatic disorder (also referred to as orthostatic intolerance) by monitoring the patient's orthostatic health metrics. Orthostatic disorder includes orthostatic hypotension (OH), neurogenic orthostatic hypotension (nOH), non-neurogenic orthostatic hypotension, initial orthostatic hypotension (IOH), delayed orthostatic hypotension (dOH), sustained orthostatic hypotension, supine hypertension, postural orthostatic tachycardia syndrome (POTS), and dysautonomia.

### BACKGROUND

Orthostatic hypotension (OH) is a condition where a person's blood pressure drops significantly when they move from lying down or sitting to standing up. This sudden drop in blood pressure can cause dizziness, lightheadedness, blurred vision, fainting, and even falls. It occurs because the body struggles to adjust blood flow quickly enough to maintain stable pressure when the position changes.

Orthostatic hypotension disproportionately affects the elderly, exacerbated by multiple age-related factors that impair the body's orthostatic response, along with the common use of OH-inducing medications such as antihypertensives, antidepressants, and analgesics. Orthostatic hypotension is present in approximately 25-30% of the older adult population and affects up to 70% of nursing home residents. Orthostatic hypotension is recognized as an independent risk factor for falls in older adults, with studies indicating a 2.5-fold increased risk of falls. Consequently, major medical organizations in North America and Europe recommend monitoring orthostatic vital signs (OVS) in older adults who experience syncope or OH-related symptoms. Additionally, the Centers for Disease Control and Prevention (CDC) emphasize the importance of managing orthostatic hypotension as a critical, evidence-based intervention for fall prevention. It is recommended that OVS screening be integrated into comprehensive geriatric assessment protocols, both in hospital settings and for home care.

### SUMMARY

According to a first aspect, a method (e.g., for detecting orthostatic vital signs such as for diagnosing an orthostatic disorder) is disclosed. The method involves continuously generating photoplethysmogram (PPG) and motion data during a stand-up or sit-up evaluation. It associates the PPG data with a first phase when the user is detected in a supine position and with a third phase when the user is standing or sitting up. The method determines one or more orthostatic metrics based on the PPG data from both phases.

According to a second aspect, a device is disclosed. The device includes one or more processors and a memory storing instructions that, when executed by the one or more processors, cause the monitoring device to: obtain photoplethysmogram (PPG) data and motion data; in response to determination, based on the motion data, that an angle associated with a user's posture indicates that the user is in a supine position, associate the PPG data with a first phase; in response to determination, based on the motion data, that the angle associated with the user's posture indicates that the user is in a standing up position or a sitting up position, associate the PPG data with a third phase; and determine one or more orthostatic metrics based on the PPG data associated with the first phase and the PPG data associated with the third phase.

According to a third aspect, there is disclosed a non-transitory computer-readable medium comprising instructions that, when executed by one or more processors of a device, cause the deice to: generate photoplethysmogram (PPG) data and motion data continuously; in response to determination, based on the motion data, that an angle associated with a user's posture indicates that the user is in a supine position, associate the PPG data with a first phase; in response to determination, based on the motion data, that the angle associated with the user's posture indicates that the user is in a standing up position or a sitting up position, associate the PPG data with a third phase; and determine one or more orthostatic metrics based on the PPG data associated with the first phase and the PPG data associated with the third phase.

According to a fourth aspect, there is disclosed a system including a monitoring device and a computing device. The monitoring device including one or more first processors and first memory storing first instructions that, when executed by the one or more first processors, cause the monitoring device to: generate photoplethysmogram (PPG) data and motion data continuously; in response to determination, based on the motion data, that an angle associated with a user's posture indicates that the user is in a supine position, associate the PPG data with a first phase; in response to determination, based on the motion data, that the angle associated with the user's posture indicates that the user is in a standing up position or a sitting up position, associate the PPG data with a third phase; determine one or more orthostatic metrics based on the PPG data associated with the first phase and the PPG data associated with the third phase; providing the one or more orthostatic metrics to a computing device. The computing device including one or more second processors and second memory storing first instructions that, when executed by the one or more second processors, cause the computing device to: store the one or more orthostatic metrics form the monitoring device. The system may be configured to perform the method(s) described herein.

According to a fifth aspect, there is disclosed a system having one or more processors, the system configured to: obtain photoplethysmogram (PPG) data and motion data, which PPG data and motion data may be monitored continuously; in response to determination, based on the motion data, that an angle associated with a user's posture indicates that the user is in a supine position, associate the PPG data with a first phase; in response to determination, based on the motion data, that the angle associated with the user's posture indicates that the user is in a standing up position or a sitting up position, associate the PPG data with a third phase; and determine one or more orthostatic metrics based on the PPG data associated with the first phase and the PPG data associated with the third phase. The system may be further configured to store the one or more orthostatic metrics for further evaluation. The system may be configured to perform the method(s) described herein.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The claimed subject matter is not limited to implementations that solve any or all disadvantages noted in the Background.

The details of one or more implementations of the disclosure are set forth in the accompanying drawings and the description below. Other aspects, features, and advantages will be apparent from the description and drawings, and from the claims.

### DESCRIPTION OF DRAWINGS

FIG. 1 includes a schematic view of a system for monitoring patient's orthostatic health metrics in accordance with some implementations of this disclosure.

FIG. 2 includes a schematic view of a patient monitor for monitoring patient's orthostatic health metrics in accordance with some implementation of this disclosure.

FIG. 3-1 and FIG. 3-2 include schematic view of a system for monitoring patient's orthostatic health metrics in accordance with some implementations of this disclosure.

FIG. 4-1 and FIG. 4-2 includes schematic view of a system for monitoring patient's orthostatic health metrics in accordance with some implementations of this disclosure.

FIG. 5 includes a flowchart of an example arrangement of operations for a method for determining an orthostatic metric value associated with orthostatic time constant (OTC) in accordance with some implementations of this disclosure.

FIG. 6-1 and FIG. 6-2 include a flowchart of an example arrangement of operations for a method for determining an orthostatic metric value associated with an orthostatic pulse volume reduction (OPVR) in accordance with some implementations of this disclosure.

FIG. 7 includes a flowchart of an example arrangement of operations for a method for determining an orthostatic metric value associated with postural orthostatic tachycardia pulse volume reduction (POT) in accordance with some implementations of this disclosure.

FIG. 8 includes a flowchart of an example arrangement of operations for a method for determining an orthostatic metric value associated with a first orthostatic hypovolemia (OHV1) in accordance with some implementations of this disclosure.

FIG. 9 includes a flowchart of an example arrangement of operations for a method for determining an orthostatic metric value associated with a second orthostatic hypovolemia (OHV2) in accordance with some implementations of this disclosure.

FIG. 10 includes a flowchart of an example arrangement of operations for a method for monitoring orthostatic metrics in a standup test in accordance with some implementations of this disclosure.

FIG. 11 includes a schematic view of a system for training a model for diagnosing orthostatic disorder.

FIG. 12 includes a schematic view of an example computing device that may be used to implement the systems and methods described herein.

Like reference symbols in the various drawings indicate like elements.

### DETAILED DESCRIPTION

Despite the strong clinical need for monitoring orthostatic hypotension (OH), point-of-care diagnostic technologies remain limited. In homes and most clinical settings, the prevailing method for OH diagnosis is postural blood pressure measurement using a BP cuff. The clinical consensus defines OH as a sustained drop in systolic BP of at least 20 mm Hg within 3 minutes of standing or postural angle change. However, the hemodynamic response to upright posture changes-such as increased heart rate, cardiac output, and vasoconstriction-typically occurs within the first 10-30 seconds of standing. As a result, conventional diagnostics fail to capture the critical initial orthostatic response. This disclosure presents an affordable wearable remote patient monitoring device and wearable remote patient monitoring system designed to capture this critical initial orthostatic response (e.g., orthostatic vital signs within the first 10-30 seconds of standing) that cannot be detected by traditional blood pressure cuff methods.

Described herein is a system, device, and method of analyzing photoplethysmography (PPG) waveforms collected (e.g., sensed) above a person's shoulders (e.g., neck, head, earlobe) while the person transitions from one postural position to the other. Examples of transitions include supine to standing, sit to standing, supine to sit, etc. By analyzing the PPG waveform and extracting orthostatic metrics (e.g., orthostatic vital signs (OVS)), as discussed below, a number of potential diseases may be diagnosed including cardiovascular and autonomic disorders. Pursuant to an implementation, the system, device, and method may be used to diagnose orthostatic disorders, including but not limited to orthostatic hypotension (OH), initial orthostatic hypotension (IOH), delayed orthostatic hypotension, sustained orthostatic hypotension, neurogenic orthostatic hypotension, non-neurogenic orthostatic hypotension, supine hypertension, orthostatic hypertension, postural orthostatic tachycardia syndrome (POTS), and dysautonomia.

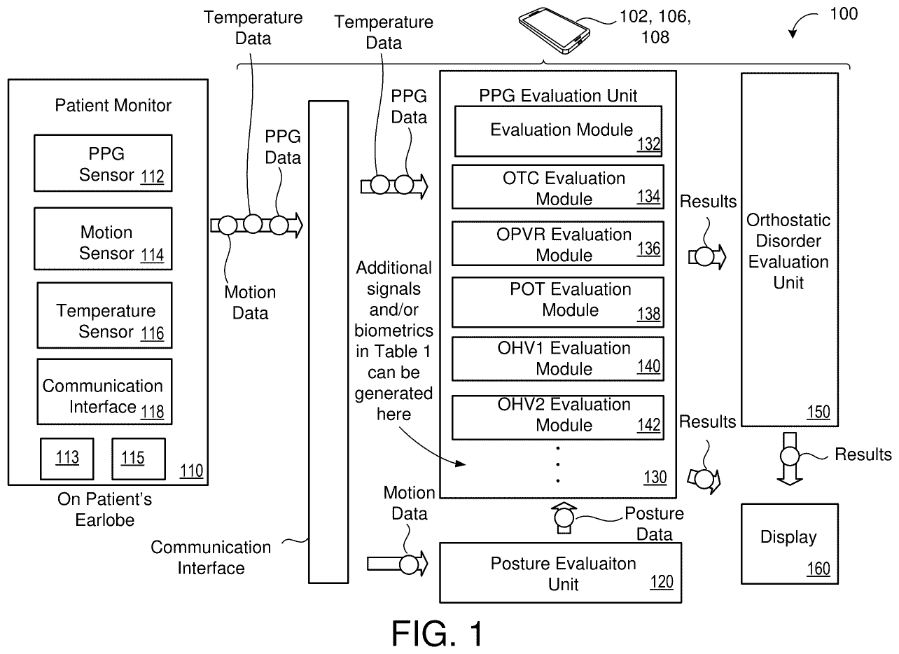

Referring to FIG. 1, in some implementations, an example system 100 includes a computing device 102 and a patient monitor 110 (e.g., blood volume monitor) associated with the computing device 102. The computing device 102 may be a computer or smart device such as smartphone including computing resources 106 (e.g., data processing hardware) and storage resources 108 (e.g., memory hardware). The patient monitor 110 may be a computer including computing resources 113 and storage resources 115 necessary for its operation.

The patient monitor 110 is configured with a photoplethysmogram (PPG) sensor 112, which measures blood volume (and/or blood flow and/or blood oxygen level) at suitable body part of the patient (e.g., upper extremity body part such as the earlobe, forehead, or neck) and generate PPG data based on the measurement. The patient monitor 110 may be a device configured to measure vitals above the shoulder region of a person as described herein, including for example the sensor and method of continuous health monitoring as described in co-owned U.S. patent application Ser. No. 16/284,329 filed Feb. 25, 2019, now issued as U.S. Pat. No. 11,330,993, the contents of which are hereby incorporated by reference in its entirety. The blood volume measured at these upper extremity body parts is related to the blood volume in the head of the patient (e.g., brain). Accordingly, the PPG data can be used to estimate the blood volume in the head of the patient. The patient monitor 110 may also include a temperature sensor 116 to account for thermoregulatory variations in blood flow. For example, the patient monitor 110 may adjust (e.g., calibrate, compensate) the PPG data (e.g., PPG signal, PPG waveform) generated from the PPG sensor 112 based on the temperature readings from the temperature sensor 116, using a model that maps temperature to expected or anticipated changes in the PPG data. As a result, the PPG data in FIG. 1 may be adjusted or calibrated PPG data. In some implementations, the patient monitor 110 transmits the PPG data (that is not adjusted based on the temperature) and temperature data (including the temperature readings from the temperature sensor 116) to the computing device 102 In this example, as shown in FIG. 1, the computing device 102 may then adjust (e.g., calibrate, compensate) the PPG data (e.g., PPG signal, PPG waveform) generated from the PPG sensor 112 based on the temperature readings from the temperature sensor 116. This adjustment may be performed using a model that maps temperature to expected or anticipated changes in the PPG data. The temperature data obtained from temperature sensor 116 can be utilized to assess the patient's stress level, as body temperature tends to decrease when the body is experiencing stress.

Additionally, the patient monitor 110 includes a motion sensor 114 (e.g., accelerometer) configured to generate motion data (e.g., acceleration in X-axis, Y-axis, and Z-axis) of the patient. The patient monitor 110 includes a communication interface 118 (e.g., Bluetooth interface) that enables the transmission of data collected by the PPG sensor 112, the motion sensor 114, and the temperature sensor 116 (e.g., motion data, PPG data, temperature data) to the computing device 102. In this example, the computing device 102 is a patient's computing device (e.g., smartphone).

The computing device 102 is configured to receive patient data, such as the PPG data, the temperature data and motion data, from the patient monitor 110 (through its communication interface, such as Bluetooth interface). Based on this input, the computing device 102 generates one or more orthostatic metrics, which will be further detailed in subsequent sections of this disclosure.

In some implementations, the computing device 102 is further configured to detect changes in posture (e.g., changes in the head angle of the patient, changes in the torso angle of the patient) or movement of the patient, such as the transition from lying down to standing or from lying down to sitting, based on the motion data from the patient monitor 110. This configuration allows for the correlation of posture changes with (real-time) blood volume data and other data derived from the (real-time) blood volume data (e.g., heart rate waveform, pulse waveform, pulse volume waveform, pulse amplitude waveform, difference amplitude waveform, difference amplitude waveform) enabling more accurate monitoring of the patient's orthostatic response.

The computing device 102 includes a posture evaluation unit 120 configured to detect changes in posture or movement of the patient, such as transitions from lying down to standing or from lying down to sitting, based on the motion data from the patient monitor 110 in accordance with some implementations. For instance, the posture evaluation unit 120 determines the tilt angle of the patient monitor 110 with respect to the ground based on the motion data from the motion sensor. When the patient monitor 110 is worn at the earlobe of the patient (or around the head, for example being held in place by a hat), the posture evaluation unit can determine the tilt angle of the patient's head (or the tilt angle of the patient's torso) in relation to the ground. Based on this tilt angle, the posture evaluation unit 120 determines the patient's posture. For example, when the patient's head is approximately at 0°, the posture evaluation unit 120 determines that the patient is lying down. When the head is approximately at 90°, the posture evaluation unit 120 determines that the patient is standing up. A change in head position from approximately 0° to 90° indicates that the patient is in the process of transitioning to a standing (or sitting upright) posture. In some implementations, the posture evaluation unit 120 generates posture data, including the start time and end time for each posture duration: the first phase when the patient is lying down, the second phase while the patient is in the process of standing up (or sitting upright), and the third phase when the patient is standing up.

In some implementations, the patient monitor 110 includes the posture evaluation unit 120. In this case, the patient monitor 110 transmits the posture data to the computing device 102 in lieu of the motion data. In some implementations, the patient monitor 110 transmits both the motion data and the posture data to the computing device 102.

In some implementations, the computing device 120 includes PPG evaluation unit 130. As described above, in some implementations, the PPG evaluation unit 130 configured to adjust (e.g., calibrate, compensate) the PPG data (e.g., PPG signal, PPG waveform) generated from the PPG sensor 112 based on the temperature readings from the temperature sensor 116. As a result, the PPG data provided to the PPG evaluation 130 is adjusted, even if the PPG data was not previously adjusted by the patient monitor 110. The PPG evaluation unit 130 includes one or more evaluation modules 132-142. In some implementations, the evaluation unit 130 includes a general evaluation module 132 configured to generate one or more biometric waveforms (e.g. heart rate waveform, pulse amplitude waveform, pulse volume waveform, difference amplitude waveform) from PPG waveform of the PPG data and posture data and generate monitoring data.

In some implementations, the PPG evaluation unit 130 includes orthostatic time constant (OTC) evaluation module 134. The OTC evaluation module 134 determines OTC value. In some implementations, OTC value is a time between a start time of the second phase (e.g., ending time of the first phase) and a time the patient reaches a stabilized condition (e.g., in a condition recovered from OH) in the third phase based on one or more biometrics (e.g., blood volume, heart rate, difference amplitude, interbeat interval, pulse volume).

In some implementations, the OTC evaluation module 134 analyzes PPG waveform in the PPG data corresponding to (e.g., associated with) the third phase and determines a time when the PPG waveform becomes stabilized (e.g., blood volume fluctuates within a predetermined % value such as 10%). In some implementations, the OTC evaluation module 134 analyzes PPG waveform in the PPG data corresponding to (e.g., associated with) the third phase and determine a time when the heart rate derived from the PPG waveform becomes stabilized (e.g., heart rate fluctuates within a predetermined % value such as 10%). In some implementations, the OTC evaluation module 134 analyzes PPG waveform in the PPG data corresponding to (e.g., associated with) the third phase and determine a time when the pulse amplitude derived from the PPG waveform becomes stabilized (e.g., heart rate fluctuates within a predetermined % value such as 10%). In some implementations, the OTC evaluation module 134 analyzes PPG waveform in the PPG data corresponding to (e.g., associated with) the third phase and determine a time when the difference amplitude derived from the PPG waveform becomes stabilized (e.g., difference amplitude fluctuates within a predetermined % value such as 10%). In some implementations, the OTC evaluation module 134 analyzes PPG waveform in the PPG data corresponding to (e.g., associated with) the third phase and determines a time when the interbeat interval derived from the PPG waveform becomes stabilized (e.g., interbeat interval fluctuates within a predetermined % value such as 10%). In some implementations, the OTC evaluation module 134 analyzes PPG waveform in the PPG data corresponding to (e.g., associated with) the third phase and determines a time when the pulse volume derived from the PPG waveform becomes stabilized (e.g., pulse volume fluctuates within a predetermined % value such as 10%).

In some implementations, the OTC evaluation module 134 determine OTC value based on the time between the start time of the second phase and the determined time the patient is stabilized. In some implementations, the OTC evaluation module 134 determine OTC value based on a time between the start time of the third phase and the determined time the patient is stabilized in the third phase.

In some implementations, the PPG evaluation unit 130 includes an orthostatic pulse volume reduction (OPVR) evaluation module 136. The OPVR evaluation module 136 determine the percentage reduction in pulse volume upon standing or sitting upright, based on the PPG data and posture data. The OPVR percentage value serves as an indicator of the percentage drop in blood volume in the patient's head (e.g., brain).

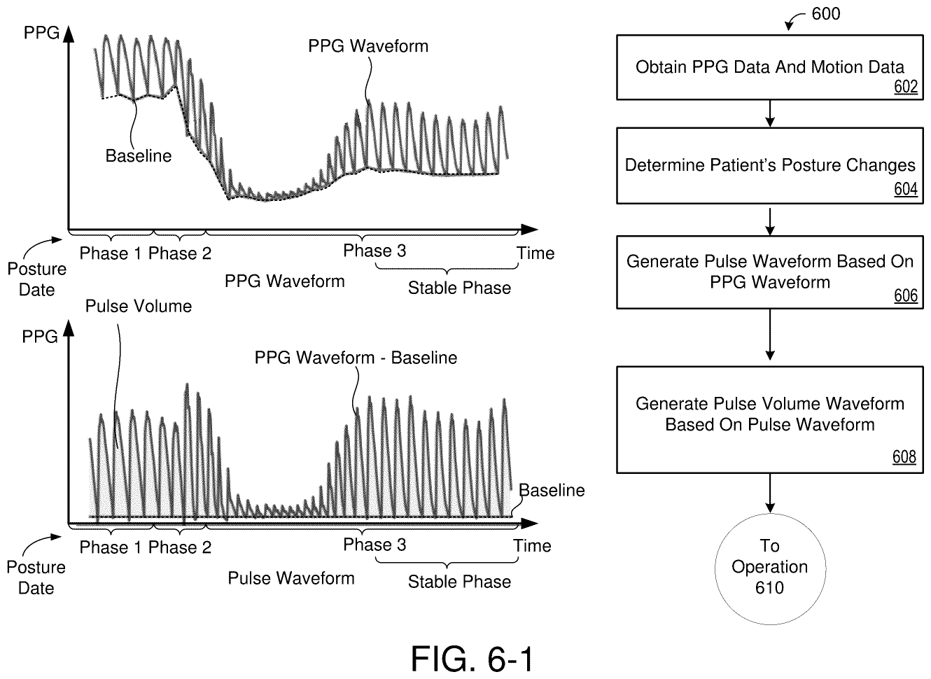

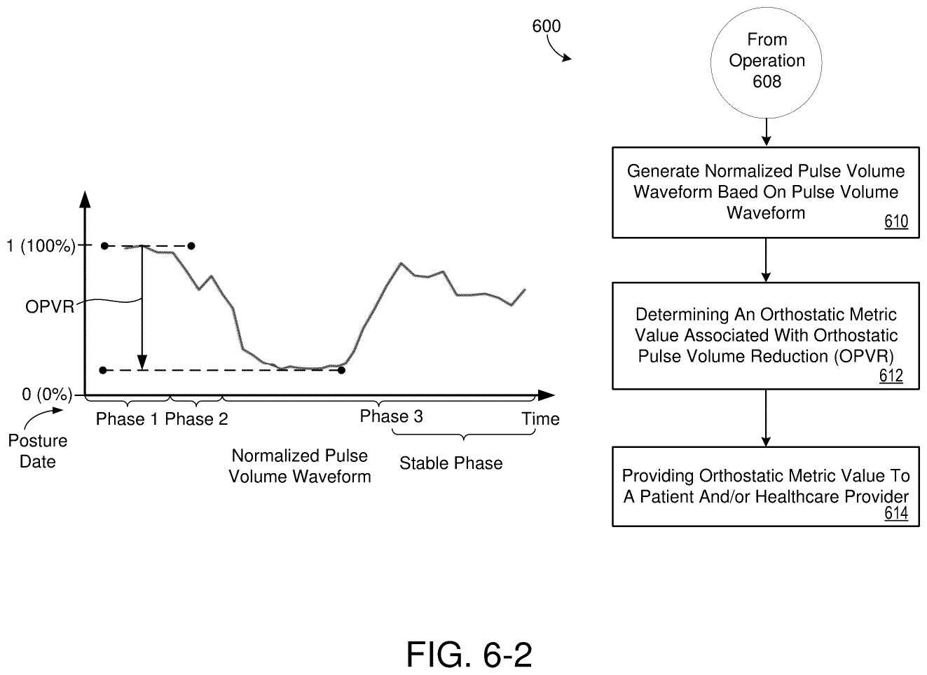

To determine the OPVR percentage value, the OPVR evaluation module 136 generates a pulse waveform based on the PPG waveform in the PPG data (for example, by subtracting the baseline PPG signal as shown in FIG. 6-1), calculates a pulse volume for each pulse in the pulse waveform (for example, by integrating the area under the curve for each pulse), and generates a pulse volume waveform based on the pulse volume calculated for each pulse. The OPVR evaluation module 136 further normalizes the pulse volume waveform by dividing the pulse volume waveform with a maximum pulse volume value in the first phase and determine the percentage reduction in pulse volume based on a normalized maximum pulse volume value in the first phase and a normalized minimum pulse volume value in the third phase. OPVR evaluation module 136 generates OPVR data based on the determined OPVR percentage value.

In some implementations, the PPG evaluation unit 130 includes a postural orthostatic tachycardia pulse volume reduction (POT) evaluation module 138. POT evaluation module 138 determines the POT value which indicates an increase in heart rate upon standing—this increase is a symptom of postural orthostatic tachycardia syndrome.

The POT evaluation module 138 can correlate PPG data with the posture changes for determining the POT value. For example, in some implementations, the POT evaluation module 138 analyzes the PPG waveform within the PPG data to derive the heart rate waveform and determine the increased heart rate (e.g., maximum difference in heart rate) based on a heart rate value (e.g., minimum heart rate) in the first phase and a heart rate value (e.g., maximum heart rate) in the third phase. POT evaluation module 138 generates POT data based on the determined POT value.

In some implementations, the PPG evaluation unit 130 includes a first orthostatic hypovolemia (OHV1) evaluation module 140. The OHV1 evaluation module 140 determines drop in blood volume upon standing or sitting upright, based on the PPG data and posture data. The OHV1 value serves as an indicator of the drop in blood volume in the patient's head (e.g., brain).

To determine the OHV1 value, the OHV1 evaluation module 140 determines a blood volume value (e.g., minimum blood volume value) in the first phase and a blood volume value (e.g., minimum blood volume value) in the third phase based on the waveform in the PPG data and posture data. The OHV1 evaluation module 140 further determined the difference between the blood volume value (e.g., minimum blood volume value) in the first phase and the blood volume (e.g., minimum blood volume value) in the third phase to determine OHV1 value. OHV1 evaluation module 140 generates OHV1 data based on the determined OHV1 value.

In some implementations, the PPG evaluation unit 130 includes a second orthostatic hypovolemia (OHV2) evaluation module 142. The OHV2 evaluation module 142 determines sustained reduction in blood volume based on the PPG data and posture data.

To determine the OHV2 value, the OHV2 evaluation module 142 determines a time the patient reaches a stabilized condition (e.g., in a condition recovered from OH) in the third phase based on one or more biometrics (e.g., blood volume, heart rate, difference amplitude, interbeat interval, pulse volume).

In some implementations, the OHV2 evaluation module 142 analyzes PPG waveform in the PPG data corresponding to (e.g., associated with) the third phase and determine a time when the PPG waveform becomes stabilized (e.g., blood volume fluctuates within a predetermined % value such as 10%). In some implementations, the OHV2 evaluation module 142 analyzes PPG waveform in the PPG data corresponding to (e.g., associated with) the third phase and determine a time when the heart rate derived from the PPG waveform becomes stabilized (e.g., heart rate fluctuates within a predetermined % value such as 10%). In some implementations, the OHV2 evaluation module 142 analyzes PPG waveform in the PPG data corresponding to (e.g., associated with) the third phase and determine a time when the pulse amplitude derived from the PPG waveform becomes stabilized (e.g., heart rate fluctuates within a predetermined % value such as 10%). In some implementations, the OHV2 evaluation module 142 analyzes PPG waveform in the PPG data corresponding to (e.g., associated with) the third phase and determine a time when the difference amplitude derived from the PPG waveform becomes stabilized (e.g., difference amplitude fluctuates within a predetermined % value such as 10%). In some implementations, the OHV2 evaluation module 142 analyzes PPG waveform in the PPG data corresponding to (e.g., associated with) the third phase and determine a time when the interbeat interval derived from the PPG waveform becomes stabilized (e.g., interbeat interval fluctuates within a predetermined % value such as 10%). In some implementations, the OHV2 evaluation module 142 analyzes PPG waveform in the PPG data corresponding to (e.g., associated with) the third phase and determine a time when the pulse volume derived from the PPG waveform becomes stabilized (e.g., pulse volume fluctuates within a predetermined % value such as 10%).

The OHV2 evaluation module 142 determines the blood volume value (e.g., minimum blood volume value) during the first phase from the PPG data and the blood volume value (e.g., minimum blood volume value) after the patient reaches a stabilized condition. The OHV2 evaluation module 142 further calculates the difference between the determined blood volume value in the first phase and the determined blood volume value after the patient is in a stabilized condition in the third phase to determine the OHV2 value. The OHV2 evaluation module 142 then generates OHV2 data based on the calculated OHV2 value.

In some implementations, the PPG evaluation unit 130 provides one or more results (e.g., orthostatic metrics such as POT value, OTC value, OHV1 value, OHV2 value, OPVR % value) to the patient in any suitable method. For example, in some implementations, the computing device 102 outputs the results to a display 160.

In some implementations, the PPG evaluation unit 130 provide the PPG data (e.g., PPG waveform) and/or other data derived from the PPG data such as monitoring data (e.g., heart rate waveform, pulse waveform, pulse volume waveform, pulse amplitude waveform, difference amplitude waveform) to the patient in any suitable method. For example, in some implementations, the computing device 102 outputs the data (e.g., one or more of the aforementioned waveforms) to the display.

In some implementations, the PPG evaluation unit 130 provides one or more results (e.g., orthostatic metrics such as POT value, OTC value, OHV1 value, OHV2 value, OPVR % value) to the patient's healthcare provider in any suitable method. For example, in some implementations, the computing device 102 transmits the results to the health care provider.

In some implementations, the PPG evaluation unit 130 provide the PPG data (e.g., PPG waveform) and/or other data derived from the PPG data (e.g., heart rate waveform, pulse waveform, pulse volume waveform, pulse amplitude waveform, difference amplitude waveform) to the patient's healthcare provider in any suitable method. For example, in some implementations, the computing device transmits the results to the healthcare provider.

In some implementations, the computing device includes orthostatic disorder evaluation unit 150 configured to determine whether the patient is more likely to having the orthostatic disorder based on one or more results (e.g., orthostatic metrics such as POT value, OTC value, OHV1 value, OHV2 value, OPVR % value) from the PPG evaluation unit.

For example, in some implementations, when the orthostatic disorder evaluation unit 150 determines that the OTC value from the PPG evaluation unit 130 is equal to or greater than a predetermined value, it notifies the patient that they are more likely to have orthostatic hypotension (OH). Similarly, when the POT value is equal to or less than a predetermined value, the orthostatic disorder evaluation unit 150 notifies the patient of an increased likelihood of having dysautonomia or impaired autonomic response. Additionally, if the OHV1 or OHV2 values are equal to or greater than their respective thresholds, the orthostatic disorder evaluation unit 150 alerts the patient to the likelihood of having the initial or sustained orthostatic hypotension, respectively. The notification can be provided in any suitable manner, such as through a display message on the display 160. Additionally, in some cases, multiple metrics may be used to make a diagnosis. For example, if OHV1 is larger and OHV2 is smaller than a predetermined threshold, the system may inform the patient of initial orthostatic hypotension (IOH). Another example is if OHV1 is smaller and POT is larger than their respective thresholds, the system may diagnose postural orthostatic tachycardia syndrome (POTS).

In some implementations, the computing device 102 transmits the notification to the health care provider.

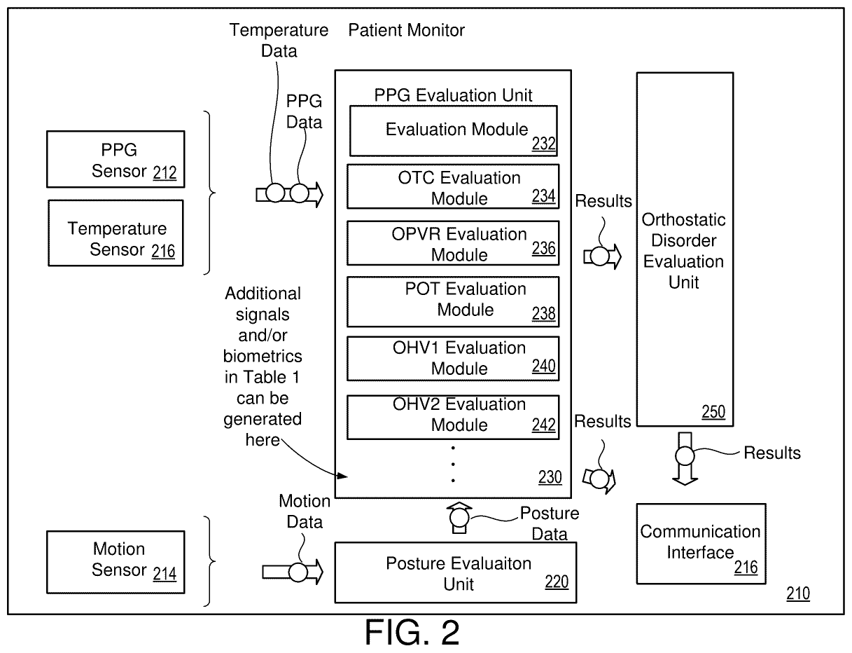

Referring to FIG. 2, in some implementations, an example patient monitor 210 (e.g., blood volume monitor). In some implementations, the patient monitor 210 is associated with a computing device (e.g., patient's computing device). The patient monitor 210 may be a computer including computing resources and storage resources necessary for its operation.

The patient monitor 210 is configured with a photoplethysmogram (PPG) sensor 212, which measures blood volume (and/or blood flow and/or blood oxygen level) at suitable body part of the patient (e.g., upper extremity body part such as the earlobe, forehead, or neck) and generate PPG data based on the measurement. The patient monitor 210 and/or sensor 212 may be a device configured to measure vitals above the shoulder region of a person as described herein, including for example the sensor and method of continuous health monitoring as described in co-owned U.S. patent application Ser. No. 16/284,329 filed Feb. 25, 2019, now issued as U.S. Pat. No. 11,330,993, the contents of which are hereby incorporated by reference in its entirety. The blood volume measured at these upper extremity body parts is related to the blood volume in the head of the patient (e.g., brain). Accordingly, the PPG data can be used to estimate the blood volume in the head of the patient. The patient monitor 210 may also include a temperature sensor 216 to account for thermoregulatory variations in blood flow. For example, the patient monitor 210 may adjust (e.g., calibrate, compensate) the PPG data (e.g., PPG signal, PPG waveform) generated from the PPG sensor 212 based on the temperature readings from the temperature sensor 216, using a model that maps temperature to expected or anticipated changes in the PPG data. As a result, the PPG data in FIG. 2 may be adjusted or calibrated PPG data. The temperature data obtained from temperature sensor 216 can be utilized to assess the patient's stress level, as body temperature tends to decrease when the body is experiencing stress.

Additionally, the patient monitor 210 includes a motion sensor 214 (e.g., accelerometer) configured to generate motion data (e.g., acceleration in X-axis, Y-axis, and Z-axis) of the patient.

Based on input (e.g., patient data, such as the PPG data, the temperature data, and motion data), the patient monitor 210 generates one or more orthostatic metrics, which will be further detailed in subsequent sections of this disclosure.

In some implementations, the patient monitor 210 is further configured to detect changes in posture (e.g., changes in the head angle of the patient, changes in the torso angle of the patient) or movement of the patient, such as the transition from lying down to standing or from lying down to sitting, based on the motion data from the motion sensor 214. This configuration allows for the correlation of posture changes with (real-time) blood volume data and other data derived from the (real-time) blood volume data (e.g., heart rate waveform, pulse waveform, pulse volume waveform, pulse amplitude waveform, difference amplitude waveform, difference amplitude waveform) enabling more accurate monitoring of the patient's orthostatic response.

The patient monitor 210 includes a posture evaluation unit 220 configured to detect changes in posture or movement of the patient, such as transitions from lying down to standing or from lying down to sitting, based on the motion data from the motion sensor 214 in accordance with some implementations. For instance, the posture evaluation unit 220 determines the tilt angle of the patient monitor 210 with respect to the ground based on the motion data from the motion sensor. When the patient monitor 210 is worn at the earlobe of the patient (or around the head, for example being held in place by a hat), the posture evaluation unit can determine the tilt angle of the patient's head (or the tilt angle of the patient's torso) in relation to the ground. Based on this tilt angle, the posture evaluation unit 220 determines the patient's posture. For example, when the patient's head is approximately at 0°, the posture evaluation unit 220 determines that the patient is lying down. When the head is approximately at 90°, the posture evaluation unit 220 determines that the patient is standing up. A change in head position from approximately 0° to 90° indicates that the patient is in the process of transitioning to a standing (or sitting upright) posture. In some implementations, the posture evaluation unit 220 generates posture data, including the start time and end time for each posture duration: the first phase when the patient is lying down, the second phase while the patient is in the process of standing up (or sitting upright), and the third phase when the patient is standing up.

In some implementations, the patient monitor includes PPG evaluation unit 230. As described above, in some implementations, the patient monitor 210 is configured to adjust (calibrate, compensate) the PPG data (e.g., PPG signal, PPG waveform) generated from the PPG sensor 212 based on the temperature readings from the temperature sensor 216. In some implementations, the adjustment to the PPG data is performed by the PPG evaluation unit 230.

The PPG evaluation unit 230 includes one or more evaluation modules 232-242. In some implementations, the evaluation unit 230 includes a general evaluation module 232 configured to generate one or more biometric waveforms (e.g. heart rate waveform, pulse amplitude waveform, pulse volume waveform, difference amplitude waveform) from PPG waveform of the PPG data and posture data and generate monitoring data.

In some implementations, the PPG evaluation unit 230 includes orthostatic time constant (OTC) evaluation module 234. The OTC evaluation module 234 determines OTC value. In some implementations, OTC value is a time between a start time of the second phase (e.g., ending time of the first phase) and a time the patient reaches a stabilized condition (e.g., in a condition recovered from OH) in the third phase based on one or more biometrics (e.g., blood volume, heart rate, difference amplitude, interbeat interval, pulse volume).

In some implementations, the OTC evaluation module 234 analyzes PPG waveform in the PPG data corresponding to (e.g., associated with) the third phase and determines a time when the PPG waveform becomes stabilized (e.g., blood volume fluctuates within a predetermined % value such as 10%). In some implementations, the OTC evaluation module 234 analyzes PPG waveform in the PPG data corresponding to (e.g., associated with) the third phase and determine a time when the heart rate derived from the PPG waveform becomes stabilized (e.g., heart rate fluctuates within a predetermined % value such as 10%). In some implementations, the OTC evaluation module 234 analyzes PPG waveform in the PPG data corresponding to (e.g., associated with) the third phase and determine a time when the pulse amplitude derived from the PPG waveform becomes stabilized (e.g., heart rate fluctuates within a predetermined % value such as 10%). In some implementations, the OTC evaluation module 234 analyzes PPG waveform in the PPG data corresponding to (e.g., associated with) the third phase and determine a time when the difference amplitude derived from the PPG waveform becomes stabilized (e.g., difference amplitude fluctuates within a predetermined % value such as 10%). In some implementations, the OTC evaluation module 234 analyzes PPG waveform in the PPG data corresponding to (e.g., associated with) the third phase and determines a time when the interbeat interval derived from the PPG waveform becomes stabilized (e.g., interbeat interval fluctuates within a predetermined % value such as 10%). In some implementations, the OTC evaluation module 234 analyzes PPG waveform in the PPG data corresponding to (e.g., associated with) the third phase and determines a time when the pulse volume derived from the PPG waveform becomes stabilized (e.g., pulse volume fluctuates within a predetermined % value such as 10%).

In some implementations, the OTC evaluation module 234 determine OTC value based on the time between the start time of the second phase and the determined time the patient is stabilized. In some implementations, the OTC evaluation module 234 determine OTC value based on a time between the start time of the third phase and the determined time the patient is stabilized.

In some implementations, the PPG evaluation unit 230 includes an orthostatic pulse volume reduction (OPVR) evaluation module 236. The OPVR evaluation module 236 determine the percentage reduction in pulse volume upon standing or sitting upright, based on the PPG data and posture data. The OPVR percentage value serves as an indicator of the percentage drop in blood volume in the patient's head (e.g., brain).

To determine the OPVR percentage value, the OPVR evaluation module 236 generates a pulse waveform based on the PPG waveform in the PPG data (for example, by subtracting the baseline PPG signal as shown in FIG. 6-1), calculates a pulse volume for each pulse in the pulse waveform (for example, by integrating the area under the curve for each pulse), and generates a pulse volume waveform based on the pulse volume calculated for each pulse. The OPVR evaluation module 236 further normalizes the pulse volume waveform by dividing the pulse volume waveform with a maximum pulse volume value in the first phase and determine the percentage reduction in pulse volume based on a normalized maximum pulse volume value in the first phase and a normalized minimum pulse volume value in the third phase. OPVR evaluation module 236 generates OPVR data based on the determined OPVR percentage value.

In some implementations, the PPG evaluation unit 230 includes a postural orthostatic tachycardia pulse volume reduction (POT) evaluation module 238. POT evaluation module 238 determines the POT value which indicates an increase in heart rate upon standing—this increase is a symptom of postural orthostatic tachycardia syndrome.

The POT evaluation module 238 can correlate PPG data with the posture changes for determining the POT value. For example, in some implementations, the POT evaluation module 238 analyzes the PPG waveform within the PPG data to derive the heart rate waveform and determine the increased heart rate (e.g., maximum difference in heart rate) based on a heart rate value (e.g., minimum heart rate) in the first phase and a heart rate value (e.g., maximum heart rate) in the third phase. POT evaluation module 238 generates POT data based on the determined POT value.

In some implementations, the PPG evaluation unit 230 includes a first orthostatic hypovolemia (OHV1) evaluation module 240. The OHV1 evaluation module 240 determines drop in blood volume upon standing or sitting upright, based on the PPG data and posture data. The OHV1 value serves as an indicator of the drop in blood volume in the patient's head (e.g., brain).

To determine the OHV1 value, the OHV1 evaluation module 240 determines a blood volume value (e.g., minimum blood volume value) in the first phase and a blood volume value (e.g., minimum blood volume value) in the third phase based on the waveform in the PPG data and posture data. The OHV1 evaluation module 240 further determined the difference between the blood volume value (e.g., minimum blood volume value) in the first phase and the blood volume (e.g., minimum blood volume value) in the third phase to determine OHV1 value. OHV1 evaluation module 240 generates OHV1 data based on the determined OHV1 value.

In some implementations, the PPG evaluation unit 230 includes a second orthostatic hypovolemia (OHV2) evaluation module 242. The OHV2 evaluation module 242 determines sustained reduction in blood volume based on the PPG data and posture data.

To determine the OHV2 value, the OHV2 evaluation module 242 determines a time the patient reaches a stabilized condition (e.g., in a condition recovered from OH) in the third phase based on one or more biometrics (e.g., blood volume, heart rate, difference amplitude, interbeat interval, pulse volume).

In some implementations, the OHV2 evaluation module 242 analyzes PPG waveform in the PPG data corresponding to (e.g., associated with) the third phase and determine a time when the PPG waveform becomes stabilized (e.g., blood volume fluctuates within a predetermined % value such as 10%). In some implementations, the OHV2 evaluation module 242 analyzes PPG waveform in the PPG data corresponding to (e.g., associated with) the third phase and determine a time when the heart rate derived from the PPG waveform becomes stabilized (e.g., heart rate fluctuates within a predetermined % value such as 10%). In some implementations, the OHV2 evaluation module 242 analyzes PPG waveform in the PPG data corresponding to (e.g., associated with) the third phase and determine a time when the pulse amplitude derived from the PPG waveform becomes stabilized (e.g., heart rate fluctuates within a predetermined % value such as 10%). In some implementations, the OHV2 evaluation module 242 analyzes PPG waveform in the PPG data corresponding to (e.g., associated with) the third phase and determine a time when the difference amplitude derived from the PPG waveform becomes stabilized (e.g., difference amplitude fluctuates within a predetermined % value such as 10%). In some implementations, the OHV2 evaluation module 242 analyzes PPG waveform in the PPG data corresponding to (e.g., associated with) the third phase and determine a time when the interbeat interval derived from the PPG waveform becomes stabilized (e.g., interbeat interval fluctuates within a predetermined % value such as 10%). In some implementations, the OHV2 evaluation module 242 analyzes PPG waveform in the PPG data corresponding to (e.g., associated with) the third phase and determine a time when the pulse volume derived from the PPG waveform becomes stabilized (e.g., pulse volume fluctuates within a predetermined % value such as 10%).

The OHV2 evaluation module 242 determines the blood volume value (e.g., minimum blood volume value) during the first phase from the PPG data and the blood volume value (e.g., minimum blood volume value) after the patient reaches a stabilized condition. The OHV2 evaluation module 242 further calculates the difference between the determined blood volume value in the first phase and the determined blood volume value after the patient is in a stabilized condition in the third phase to determine the OHV2 value. The OHV2 evaluation module 242 then generates OHV2 data based on the calculated OHV2 value.

In some implementations, the PPG evaluation unit 230 provides one or more results (e.g., orthostatic metrics such as POT value, OTC value, OHV1 value, OHV2 value, OPVR % value) to the patient in any suitable method. For example, in some implementations, the PPG evaluation unit 230 causes a computing device (e.g., patient's computing device) outputting the results to its display.

In some implementations, the PPG evaluation unit 230 provide the PPG data (e.g., PPG waveform) and/or other data derived from the PPG data such as monitoring data (e.g., heart rate waveform, pulse waveform, pulse volume waveform, pulse amplitude waveform, difference amplitude waveform) to the patient in any suitable method. For example, in some implementations, the patient monitor 210 transmits the data (e.g., one or more of the aforementioned waveforms) to a computing device (e.g., patient's computing device such as smartphone) associated with the patient monitor 210 through a communication interface 216 (e.g. Bluetooth interface).

In some implementations, the PPG evaluation unit 230 provides one or more results (e.g., orthostatic metrics such as POT value, OTC value, OHV1 value, OHV2 value, OPVR % value) to the patient's healthcare provider in any suitable method. For example, in some implementations, the patient monitor 210 transmits the results to the healthcare provider through a computing device (e.g., patient's computing device such as smartphone).

In some implementations, the PPG evaluation unit 230 provide the PPG data (e.g., PPG waveform) and/or other data derived from the PPG data (e.g., heart rate waveform, pulse waveform, pulse volume waveform, pulse amplitude waveform, difference amplitude waveform) to the patient's healthcare provider in any suitable method. For example, in some implementations, the patient monitor 210 transmits the results to the healthcare provider through a computing device (e.g., patient's computing device such as smartphone).

In some implementations, the computing device includes orthostatic disorder evaluation unit 250 configured to determine whether the patient is more likely to having the orthostatic disorder based on one or more results (e.g., orthostatic metrics such as POT value, OTC value, OHV1 value, OHV2 value, OPVR % value) from the PPG evaluation unit.

For example, in some implementations, when the orthostatic disorder evaluation unit 250 determines that the OTC value from the PPG evaluation unit 230 is equal to or greater than a predetermined value, it notifies the patient that they are more likely to have orthostatic hypotension (OH). Similarly, when the POT value is equal to or less than a predetermined value, the orthostatic disorder evaluation unit 250 notifies the patient of an increased likelihood of having dysautonomia or impaired autonomic response. Additionally, if the OHV1 or OHV2 values are equal to or greater than their respective thresholds, the orthostatic disorder evaluation unit 250 alerts the patient to the likelihood of having the initial or sustained orthostatic hypotension, respectively. The notification can be provided in any suitable manner, such as through a display message on a display of the computing device (e.g., patient's computing device such as smartphone). Additionally, in some cases, multiple metrics may be used to make a diagnosis. For example, if OHV1 is larger and OHV2 is smaller than a predetermined threshold, the system may inform the patient of initial orthostatic hypotension (IOH). Another example is if OHV1 is smaller and POT is larger than their respective thresholds, the system may diagnose postural orthostatic tachycardia syndrome (POTS).

In some implementations, the patient monitor 210 transmits the notification to the healthcare provider through the computing device (e.g., patient's computing device such as smartphone)

Referring to FIG. 3-1 and FIG. 3-2, in some implementations, an example system 300 includes a patient's computing device, a patient monitor 310 (e.g., blood volume monitor) associated with the patient's computing device, and a healthcare provider's computing device. The patient's computing device may be a computer or smart device such as smartphone including computing resources (e.g., data processing hardware) and storage resources (e.g., memory hardware). The health care provider's computing device may be a computer or smart device such as smartphone including computing resources (e.g., data processing hardware) and storage resources (e.g., memory hardware). In some implementations, the example system 300 includes a server (e.g., cloud server). The server may be a computer including computing resources (e.g., data processing hardware) and storage resources (e.g., memory hardware). The patient monitor 310 may be a computer including computing resources 313 and storage resources 315 necessary for its operation. In some implementations, the server is dedicated to the patient monitor 310 (e.g., operated by the patient monitor manufacturer). In some implementations, the server is operated by the heath care provider. In some implementations, the server is operated by a third party other than the healthcare provider or the patient monitor manufacturer.

The patient monitor 310 is configured with a photoplethysmogram (PPG) sensor 312, which measures blood volume (and/or blood flow and/or blood oxygen level) at suitable body part of the patient (e.g., upper extremity body part such as the earlobe, forehead, or neck) and generate PPG data based on the measurement. The patient monitor 310 and/or sensor 312 may be a device configured to measure vitals above the shoulder region of a person as described herein, including for example the sensor and method of continuous health monitoring as described in co-owned U.S. patent application Ser. No. 16/284,329 filed Feb. 25, 2019, now issued as U.S. Pat. No. 11,330,993, the contents of which are hereby incorporated by reference in its entirety. The blood volume measured at these upper extremity body parts is related to the blood volume in the head of the patient (e.g., brain). Accordingly, the PPG data can be used to estimate the blood volume in the head of the patient. The patient monitor 310 may also include a temperature sensor 316 to account for thermoregulatory variations in blood flow. For example, the patient monitor 310 may adjust (e.g., calibrate, compensate) the PPG data (e.g., PPG signal, PPG waveform) generated from the PPG sensor 312 based on the temperature readings from the temperature sensor 316, using a model that maps temperature to expected or anticipated changes in the PPG data. As a result, the PPG data in FIG. 3-1 and FIG. 3-2 may be adjusted or calibrated PPG data.

In some implementations, as shown, the patient monitor 310 transmits the PPG data (that is not adjusted based on the temperature) and temperature data (including the temperature readings from the temperature sensor 316) to the healthcare provider's computing device through the patient's computing device and the server. In this case, as shown in FIG. 3-1 and FIG. 3-2, the healthcare provider's computing device may then adjust (e.g., calibrate, compensate) the PPG data (e.g., PPG signal, PPG waveform) generated from the PPG sensor 312 based on the temperature readings from the temperature sensor 316. This adjustment may be performed using a model that maps temperature to expected or anticipated changes in the PPG data. The temperature data obtained from temperature sensor 316 can be utilized to assess the patient's stress level, as body temperature tends to decrease when the body is experiencing stress.

Additionally, the patient monitor 310 includes a motion sensor 314 (e.g., accelerometer) configured to generate motion data (e.g., acceleration in X-axis, Y-axis, and Z-axis) of the patient. The patient monitor 310 includes a communication interface (e.g., Bluetooth interface) that enables the transmission of data collected by the PPG sensor 312, the motion sensor 314, and the temperature sensor 316 (e.g., motion data, PPG data, temperature data) to the patient's computing device.

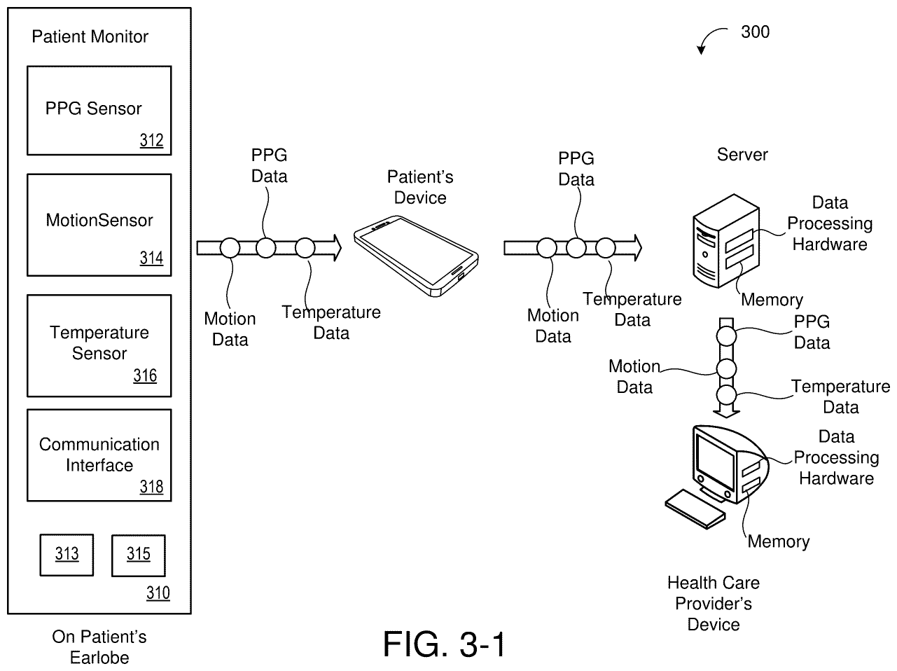

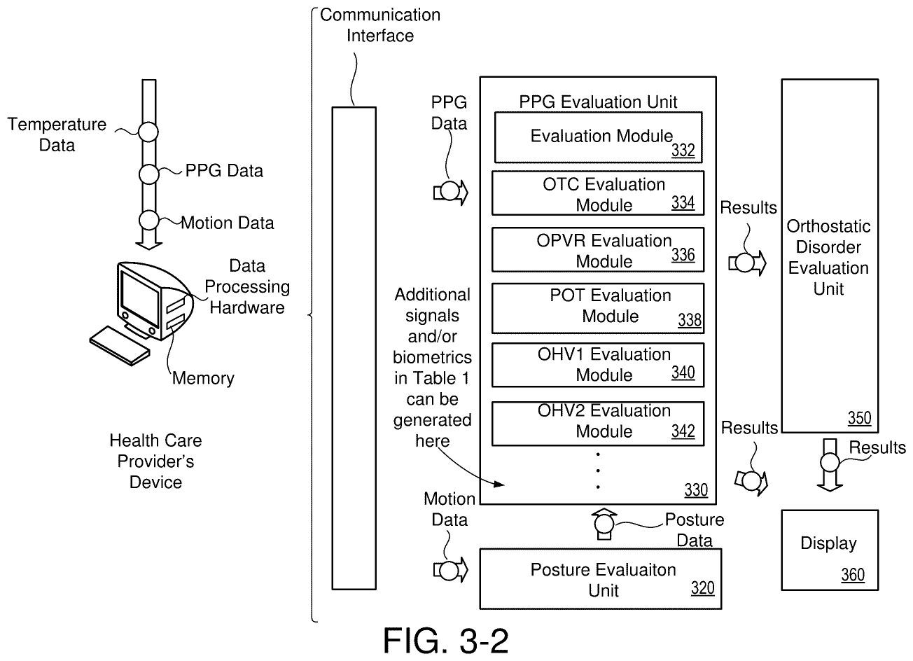

As illustrated in FIG. 3-1, the patient monitor 310 is configured to transmit the PPG data, the temperature data, and the motion data collected from the patient using any suitable methods (e.g., wireless communication such as Bluetooth communication) to the patient's computing device. The patient's computing device receives the patient data (e.g., PPG data, temperature data, the motion data) and transmits the patient data to the healthcare provider's computing device through the server. In some implementations, the patient's computing device uploads the patient data to a server (e.g., cloud server), allowing the healthcare provider's computing device to download and access the patient data.

The healthcare provider's computing device is configured to receive patient data, such as the PPG data, the temperature data, and motion data, from the patient's computing device. Based on this input, the healthcare provider's computing device generates one or more orthostatic metrics.

In some implementations, the healthcare provider's computing device is further configured to detect changes in posture (e.g., changes in the head angle of the patient, changes in the torso angle of the patient) or movement of the patient, such as the transition from lying down to standing or from lying down to sitting, based on the motion data from the patient's computing device. This configuration allows for the correlation of posture changes with (real-time) blood volume data and other data derived from the (real-time) blood volume data (e.g., heart rate waveform, pulse waveform, pulse volume waveform, pulse amplitude waveform, difference amplitude waveform, difference amplitude waveform) enabling more accurate monitoring of the patient's orthostatic response.

The healthcare provider' computing device includes a posture evaluation unit 320 configured to detect changes in posture or movement of the patient, such as transitions from lying down to standing or from lying down to sitting, based on the motion data from the patient monitor 310 in accordance with some implementations. For instance, the posture evaluation unit 320 determines the tilt angle of the patient monitor 310 with respect to the ground based on the motion data from the motion sensor. When the patient monitor 310 is worn at the earlobe of the patient (or around the head, for example being held in place by a hat), the posture evaluation unit can determine the tilt angle of the patient's head (or the tilt angle of the patient's torso) in relation to the ground. Based on this tilt angle, the posture evaluation unit 320 determines the patient's posture. For example, when the patient's head is approximately at 0°, the posture evaluation unit 320 determines that the patient is lying down. When the head is approximately at 90°, the posture evaluation unit 320 determines that the patient is standing up. A change in head position from approximately 0° to 90° indicates that the patient is in the process of transitioning to a standing (or sitting upright) posture. In some implementations, the posture evaluation unit 320 generates posture data, including the start time and end time for each posture duration: the first phase when the patient is lying down, the second phase while the patient is in the process of standing up (or sitting upright), and the third phase when the patient is standing up.

In some implementations, the patient monitor 310 includes the posture evaluation unit 320. In this case, the patient monitor 310 transmits the posture data to the healthcare provider's computing device through the patient's computing device and the server in lieu of the motion data. In some implementations, the patient monitor 310 transmits both the motion data and the posture data to the healthcare provider's computing device through the patient's computing device and the server.

In some implementations, the healthcare provider's computing device include PPG evaluation unit 330. As described above, in some implementations, the PPG evaluation unit 330 configured to adjust (e.g., calibrate, compensate) the PPG data (e.g., PPG signal, PPG waveform) generated from the PPG sensor 312 based on the temperature readings from the temperature sensor 316. As a result, the PPG data provided to the PPG evaluation 330 is adjusted, even if the PPG data was not previously adjusted by the patient monitor. The PPG evaluation unit 330 includes one or more evaluation modules 332-342. In some implementations, the PPG evaluation unit 230 includes a general evaluation module 332 configured to generate one or more biometric waveforms (e.g. heart rate waveform, pulse amplitude waveform, pulse volume waveform, difference amplitude waveform) from PPG waveform of the PPG data and posture data and generate monitoring data.

In some implementations, the PPG evaluation unit 330 includes orthostatic time constant (OTC) evaluation module 334. The OTC evaluation module 334 determines OTC value. In some implementations, OTC value is a time between a start time of the second phase (e.g., ending time of the first phase) and a time the patient reaches a stabilized condition (e.g., in a condition recovered from OH) in the third phase based on one or more biometrics (e.g., blood volume, heart rate, difference amplitude, interbeat interval, pulse volume).

In some implementations, the OTC evaluation module 334 analyzes PPG waveform in the PPG data corresponding to (e.g., associated with) the third phase and determine a time when the PPG waveform becomes stabilized (e.g., blood volume fluctuates within a predetermined % value such as 10%). In some implementations, the OTC evaluation module 334 analyzes PPG waveform in the PPG data corresponding to (e.g., associated with) the third phase and determine a time when the heart rate derived from the PPG waveform becomes stabilized (e.g., heart rate fluctuates within a predetermined % value such as 10%). In some implementations, the OTC evaluation module 334 analyzes PPG waveform in the PPG data corresponding to (e.g., associated with) the third phase and determine a time when the pulse amplitude derived from the PPG waveform becomes stabilized (e.g., heart rate fluctuates within a predetermined % value such as 10%). In some implementations, the OTC evaluation module 334 analyzes PPG waveform in the PPG data corresponding to (e.g., associated with) the third phase and determine a time when the difference amplitude derived from the PPG waveform becomes stabilized (e.g., difference amplitude fluctuates within a predetermined % value such as 10%). In some implementations, the OTC evaluation module 334 analyzes PPG waveform in the PPG data corresponding to (e.g., associated with) the third phase and determine a time when the interbeat interval derived from the PPG waveform becomes stabilized (e.g., interbeat interval fluctuates within a predetermined % value such as 10%). In some implementations, the OTC evaluation module 334 analyzes PPG waveform in the PPG data corresponding to (e.g., associated with) the third phase and determine a time when the pulse volume derived from the PPG waveform becomes stabilized (e.g., pulse volume fluctuates within a predetermined % value such as 10%).

In some implementations, the OTC evaluation module 334 determine OTC value based on the time between the start time of the second phase and the determined time the patient is stabilized. In some implementations, the OTC evaluation module 334 determine OTC value based on a time between the start time of the third phase and the determined time the patient is stabilized.

In some implementations, the PPG evaluation unit 330 includes an orthostatic pulse volume reduction (OPVR) evaluation module 336. The OPVR evaluation module 336 determine the percentage reduction in pulse volume upon standing or sitting upright, based on the PPG data and posture data. The OPVR percentage value serves as an indicator of the percentage drop in blood volume in the patient's head (e.g., brain).

To determine the OPVR percentage value, the OPVR evaluation module 336 generates a pulse waveform based on the PPG waveform in the PPG data (for example, by subtracting the baseline PPG signal as shown in FIG. 6-1), calculates a pulse volume for each pulse in the pulse waveform (for example, by integrating the area under the curve for each pulse), and generates a pulse volume waveform based on the pulse volume calculated for each pulse. The OPVR evaluation module 336 further normalizes the pulse volume waveform by dividing the pulse volume waveform with a maximum pulse volume value in the first phase and determine the percentage reduction in pulse volume based on a normalized maximum pulse volume value in the first phase and a normalized minimum pulse volume value in the third phase. OPVR evaluation module 336 generates OPVR data based on the determined OPVR percentage value.

In some implementations, the PPG evaluation unit 330 includes a postural orthostatic tachycardia pulse volume reduction (POT) evaluation module 338. POT evaluation module 338 determines the POT value which indicates an increase in heart rate upon standing—this increase is a symptom of postural orthostatic tachycardia syndrome.

The POT evaluation module 338 can correlate PPG data with the posture changes for determining the POT value. For example, in some implementations, the POT evaluation module 338 analyzes the PPG waveform within the PPG data to derive the heart rate waveform and determine the increased heart rate (e.g., maximum difference in heart rate) based on a heart rate value (e.g., minimum heart rate) in the first phase and a heart rate value (e.g., maximum heart rate) in the third phase. POT evaluation module 338 generates POT data based on the determined POT value.

In some implementations, the PPG evaluation unit 330 includes a first orthostatic hypovolemia (OHV1) evaluation module 340. The OHV1 evaluation module 340 determines drop in blood volume upon standing or sitting upright, based on the PPG data and posture data. The OHV1 value serves as an indicator of the drop in blood volume in the patient's head (e.g., brain).

To determine the OHV1 value, the OHV1 evaluation module 340 determines a determine a blood volume value (e.g., minimum blood volume value) in the first phase and a blood volume value (e.g., minimum blood volume value) in the third phase based on the waveform in the PPG data and posture data. The OHV1 evaluation module 340 further determined the difference between the blood volume value (e.g., minimum blood volume value) in the first phase and the blood volume (e.g., minimum blood volume value) in the third phase to determine OHV1 value. OHV1 evaluation module 340 generates OHV1 data based on the determined OHV1 value.

In some implementations, the PPG evaluation unit 330 includes a second orthostatic hypovolemia (OHV2) evaluation module 342. The OHV2 evaluation module 342 determines sustained reduction in blood volume based on the PPG data and posture data.

To determine the OHV2 value, the OHV2 evaluation module 342 determines a time the patient reaches a stabilized condition (e.g., in a condition recovered from OH) in the third phase based on one or more biometrics (e.g., blood volume, heart rate, difference amplitude, interbeat interval, pulse volume).

In some implementations, the OHV2 evaluation module 342 analyzes PPG waveform in the PPG data corresponding to (e.g., associated with) the third phase and determine a time when the PPG waveform becomes stabilized (e.g., blood volume fluctuates within a predetermined % value such as 10%). In some implementations, the OHV2 evaluation module 342 analyzes PPG waveform in the PPG data corresponding to (e.g., associated with) the third phase and determine a time when the heart rate derived from the PPG waveform becomes stabilized (e.g., heart rate fluctuates within a predetermined % value such as 10%). In some implementations, the OHV2 evaluation module 342 analyzes PPG waveform in the PPG data corresponding to (e.g., associated with) the third phase and determine a time when the pulse amplitude derived from the PPG waveform becomes stabilized (e.g., heart rate fluctuates within a predetermined % value such as 10%). In some implementations, the OHV2 evaluation module 342 analyzes PPG waveform in the PPG data corresponding to (e.g., associated with) the third phase and determine a time when the difference amplitude derived from the PPG waveform becomes stabilized (e.g., difference amplitude fluctuates within a predetermined % value such as 10%). In some implementations, the OHV2 evaluation module 342 analyzes PPG waveform in the PPG data corresponding to (e.g., associated with) the third phase and determine a time when the interbeat interval derived from the PPG waveform becomes stabilized (e.g., interbeat interval fluctuates within a predetermined % value such as 10%). In some implementations, the OHV2 evaluation module 342 analyzes PPG waveform in the PPG data corresponding to (e.g., associated with) the third phase and determine a time when the pulse volume derived from the PPG waveform becomes stabilized (e.g., pulse volume fluctuates within a predetermined % value such as 10%).

The OHV2 evaluation module 342 determines the blood volume value (e.g., minimum blood volume value) during the first phase from the PPG data and the blood volume value (e.g., minimum blood volume value) after the patient reaches a stabilized condition. The OHV2 evaluation module 342 further calculates the difference between the determined blood volume value in the first phase and the determined blood volume value after the patient is in a stabilized condition in the third phase to determine the OHV2 value. The OHV2 evaluation module 342 then generates OHV2 data based on the calculated OHV2 value.

In some implementations, the PPG evaluation unit 330 provides one or more results (e.g., orthostatic metrics such as POT value, OTC value, OHV1 value, OHV2 value, OPVR % value) to the healthcare provider in any suitable method. For example, in some implementations, the healthcare provider's computing device outputs the results to a display and/or stores the results to the patient's health record (e.g., electrical health record).

In some implementations, the PPG evaluation unit 330 provide the PPG data (e.g., PPG waveform) and/or other data derived from the PPG data such as monitoring data (e.g., heart rate waveform, pulse waveform, pulse volume waveform, pulse amplitude waveform, difference amplitude waveform) to the healthcare provider in any suitable method. For example, in some implementations, the healthcare provider's computing device outputs the data (e.g., one or more of the aforementioned waveforms) to the display and/or stores the data to the patient's health record.

In some implementations, the healthcare provider's computing device includes orthostatic disorder evaluation unit 350 configured to determine whether the patient is more likely to having the orthostatic disorder based on one or more results (e.g., orthostatic metrics such as POT value, OTC value, OHV1 value, OHV2 value, OPVR % value) from the PPG evaluation unit.

For example, in some implementations, if the orthostatic disorder evaluation unit 350 determines that the OTC value from the PPG evaluation unit is equal to or greater than a predetermined threshold, it notifies the healthcare provider that the patient is more likely to have orthostatic hypotension (OH). Similarly, if the POT value is equal to or less than a predetermined threshold, the orthostatic disorder evaluation unit 350 alerts the healthcare provider of the patient's increased likelihood of dysautonomia or impaired autonomic response. Additionally, if the OHV1 or OHV2 values meet or exceed their respective thresholds, the orthostatic disorder evaluation unit 350 notifies the healthcare provider of the patient's likelihood of having the initial or sustained orthostatic hypotension, respectively. The notification can be provided in any suitable manner, such as through a message on the display. Additionally, in some cases, multiple metrics may be used to make a diagnosis. For example, if OHV1 is larger and OHV2 is smaller than a predetermined threshold, the system may inform the patient of initial orthostatic hypotension (IOH). Another example is if OHV1 is smaller and POT is larger than their respective thresholds, the system may diagnose postural orthostatic tachycardia syndrome (POTS).

In some implementations, the healthcare provider's computing device provides the notification to the healthcare provider, such as through an on-screen notification on the healthcare provider's smartphone and/or display 360.

Referring to FIG. 4-1 and FIG. 4-2, in some implementations, an example system 400 includes a patient's computing device, a patient monitor 410 (e.g., blood volume monitor) associated with the patient's computing device, and a health care provider's computing device. The patient's computing device may be a computer or smart device such as smartphone including computing resources (e.g., data processing hardware) and storage resources (e.g., memory hardware). The healthcare provider's computing device may be a computer or smart device such as smartphone including computing resources (e.g., data processing hardware) and storage resources (e.g., memory hardware). The patient monitor 410 may be a computer including computing resources 413 and storage resources 415 necessary for its operation. The example system 400 also includes a server (e.g., cloud server). The server may be a computer including computing resources (e.g., data processing hardware) and storage resources (e.g., memory hardware). In some implementations, the server is dedicated to the patient monitor 410 (e.g., operated by the patient monitor manufacturer). In some implementations, the server is operated by the heath care provider. In some implementations, the server is operated by a third party other than the healthcare provider or the patient monitor manufacturer.

The patient monitor 410 is configured with a photoplethysmogram (PPG) sensor 412, which measures blood volume (and/or blood flow and/or blood oxygen level) at suitable body part of the patient (e.g., upper extremity body part such as the earlobe, forehead, or neck) and generate PPG data based on the measurement. The patient monitor 410 and/or sensor 412 may be a device configured to measure vitals above the shoulder region of a person as described herein, including for example the sensor and method of continuous health monitoring as described in co-owned U.S. patent application Ser. No. 16/284,329 filed Feb. 25, 2019, now issued as U.S. Pat. No. 11,330,993, the contents of which are hereby incorporated by reference in its entirety. The blood volume measured at these upper extremity body parts is related to the blood volume in the head of the patient (e.g., brain). Accordingly, the PPG data can be used to estimate the blood volume in the head of the patient. The patient monitor 410 may also include a temperature sensor 416 to account for thermoregulatory variations in blood flow. For example, the patient monitor 410 may adjust (e.g., calibrate, compensate) the PPG data (e.g., PPG signal, PPG waveform) generated from the PPG sensor 412 based on the temperature readings from the temperature sensor 416, using a model that maps temperature to expected or anticipated changes in the PPG data. As a result, the PPG data in FIG. 4-1 and FIG. 4-2 may be adjusted or calibrated PPG data. In some implementations, the patient monitor 410 transmits the PPG data (that is not adjusted based on the temperature) and temperature data (including the temperature readings from the temperature sensor 416) to the server through the patient's computing device. In this case, as shown in FIG. 4-1 and FIG. 4-2, the sever may then adjust (e.g., calibrate, compensate) the PPG data (e.g., PPG signal, PPG waveform) generated from the PPG sensor 412 based on the temperature readings from the temperature sensor 416. This adjustment may be performed using a model that maps temperature to expected or anticipated changes in the PPG data. The temperature data obtained from temperature sensor 416 can be utilized to assess the patient's stress level, as body temperature tends to decrease when the body is experiencing stress.

Additionally, the patient monitor 410 includes a motion sensor 414 (e.g., accelerometer) configured to generate motion data (e.g., acceleration in X-axis, Y-axis, and Z-axis) of the patient. The patient monitor 310 includes a communication interface 418 (e.g., Bluetooth interface) that enables the transmission of data collected by the PPG sensor 312, the motion sensor 314, and the temperature sensor 116 (e.g., motion data, PPG data, temperature data) to the patient's computing device (e.g., smartphone).

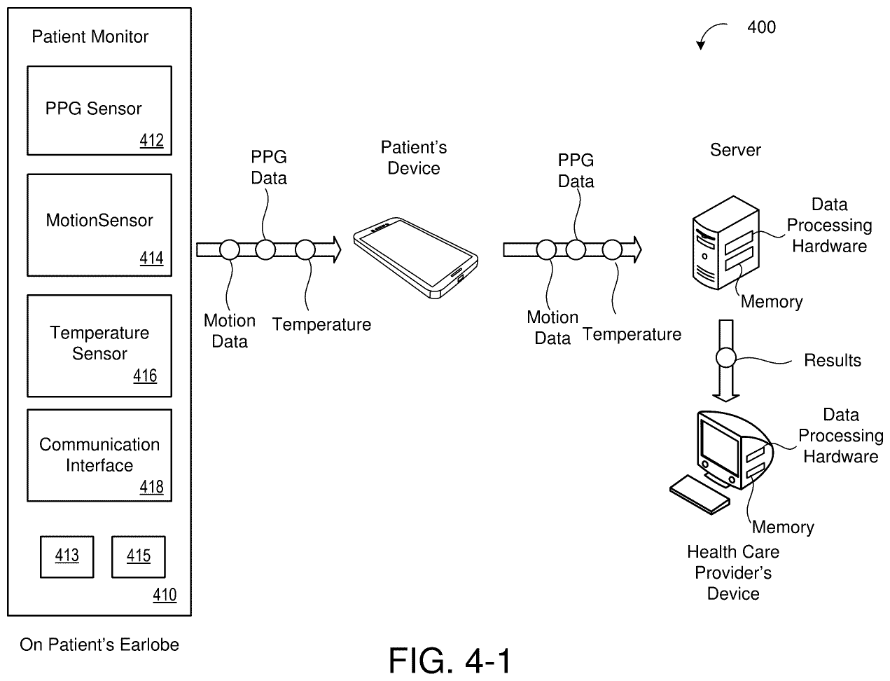

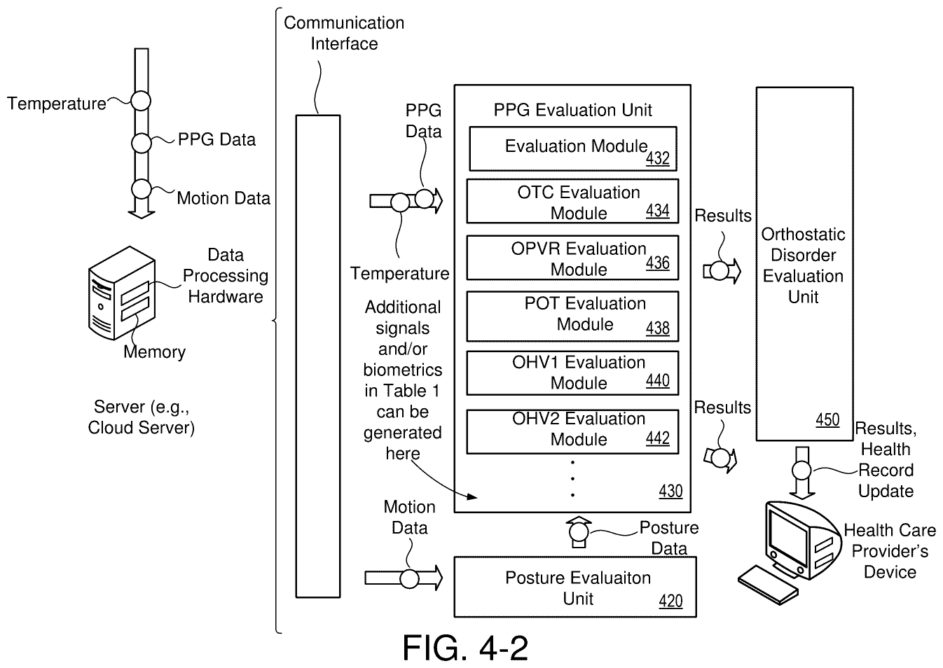

As illustrated in FIG. 4-1, the patient monitor 410 is configured to transmit the PPG data, the temperature data, and the motion data collected from the patient using any suitable methods (e.g., wireless communication such as Bluetooth communication) to the patient's computing device. The patient's computing device receives the patient data (e.g., PPG data, the motion data, temperature data) and transmits (e.g., upload) the patient data to the server.

The server is configured to receive patient data, such as the PPG data, the temperature data, and motion data, from the patient's computing device. Based on this input, the server generates one or more orthostatic metrics.

In some implementations, the server is further configured to detect changes in posture (e.g., changes in the head angle of the patient, changes in the torso angle of the patient) or movement of the patient, such as the transition from lying down to standing or from lying down to sitting, based on the motion data from the patient's computing device. This configuration allows for the correlation of posture changes with (real-time) blood volume data and other data derived from the (real-time) blood volume data (e.g., heart rate waveform, pulse waveform, pulse volume waveform, pulse amplitude waveform, difference amplitude waveform, difference amplitude waveform) enabling more accurate monitoring of the patient's orthostatic response.

The server includes a posture evaluation unit 420 configured to detect changes in posture or movement of the patient, such as transitions from lying down to standing or from lying down to sitting, based on the motion data from the patient's computing device in accordance with some implementations. For instance, the posture evaluation unit 420 determines the tilt angle of the patient monitor 410 with respect to the ground based on the motion data from the motion sensor. When the patient monitor 410 is worn at the earlobe of the patient, the posture evaluation unit can determine the tilt angle of the patient's head (or the tilt angle of the patient's torso) in relation to the ground. Based on this tilt angle, the posture evaluation unit 420 determines the patient's posture. For example, when the patient's head is approximately at 0°, the posture evaluation unit determines that the patient is lying down. When the head is approximately at 90°, the posture evaluation unit determines that the patient is standing up. A change in head position from approximately 0° to 90° indicates that the patient is in the process of transitioning to a standing (or sitting upright) posture. In some implementations, the posture evaluation unit generates posture data, including the start time and end time for each posture duration: the first phase when the patient is lying down, the second phase while the patient is in the process of standing up (or sitting upright), and the third phase when the patient is standing up.

In some implementations, the patient monitor 410 includes the posture evaluation unit 420. In this case, the patient monitor 410 transmits the posture data to the server through the patient's computing device in lieu of the motion data. In some implementations, the patient monitor 410 transmits both the motion data and the posture data the server through the patient's computing device.

In some implementations, the server includes PPG evaluation unit 430. The PPG evaluation unit 430 includes one or more evaluation modules 432-442. As described above, in some implementations, the PPG evaluation unit 430 configured to adjust (e.g., calibrate, compensate) the PPG data (e.g., PPG signal, PPG waveform) generated from the PPG sensor 412 based on the temperature readings from the temperature sensor 416. As a result, the PPG data provided to the PPG evaluation 430 is adjusted, even if the PPG data was not previously adjusted by the patient monitor. In some implementations, the PPG evaluation unit 430 includes a general evaluation module 432 configured to generate one or more biometric waveforms (e.g. heart rate waveform, pulse amplitude waveform, pulse volume waveform, difference amplitude waveform) from PPG waveform of the PPG data and posture data and generate monitoring data.

In some implementations, the PPG evaluation unit 430 includes orthostatic time constant (OTC) evaluation module 434. The OTC evaluation module 434 determines OTC value. In some implementations, OTC value is a time between a start time of the second phase (e.g., ending time of the first phase) and a time the patient reaches a stabilized condition (e.g., in a condition recovered from OH) in the third phase based on one or more biometrics (e.g., blood volume, heart rate, difference amplitude, interbeat interval, pulse volume).

In some implementations, the OTC evaluation module 434 analyzes PPG waveform in the PPG data corresponding to (e.g., associated with) the third phase and determine a time when the PPG waveform becomes stabilized (e.g., blood volume fluctuates within a predetermined % value such as 10%). In some implementations, the OTC evaluation module 434 analyzes PPG waveform in the PPG data corresponding to (e.g., associated with) the third phase and determine a time when the heart rate derived from the PPG waveform becomes stabilized (e.g., heart rate fluctuates within a predetermined % value such as 10%). In some implementations, the OTC evaluation module 434 analyzes PPG waveform in the PPG data corresponding to (e.g., associated with) the third phase and determine a time when the pulse amplitude derived from the PPG waveform becomes stabilized (e.g., heart rate fluctuates within a predetermined % value such as 10%). In some implementations, the OTC evaluation module 434 analyzes PPG waveform in the PPG data corresponding to (e.g., associated with) the third phase and determine a time when the difference amplitude derived from the PPG waveform becomes stabilized (e.g., difference amplitude fluctuates within a predetermined % value such as 10%). In some implementations, the OTC evaluation module 434 analyzes PPG waveform in the PPG data corresponding to (e.g., associated with) the third phase and determine a time when the interbeat interval derived from the PPG waveform becomes stabilized (e.g., interbeat interval fluctuates within a predetermined % value such as 10%). In some implementations, the OTC evaluation module 434 analyzes PPG waveform in the PPG data corresponding to (e.g., associated with) the third phase and determine a time when the pulse volume derived from the PPG waveform becomes stabilized (e.g., pulse volume fluctuates within a predetermined % value such as 10%).

In some implementations, the OTC evaluation module 434 determine OTC value based on the time between the start time of the second phase and the determined time the patient is stabilized. In some implementations, the OTC evaluation module 434 determine OTC value based on a time between the start time of the third phase and the determined time the patient is stabilized.

In some implementations, the PPG evaluation unit 430 includes an orthostatic pulse volume reduction (OPVR) evaluation module 436. The OPVR evaluation module 436 determine the percentage reduction in pulse volume upon standing or sitting upright, based on the PPG data and posture data. The OPVR percentage value serves as an indicator of the percentage drop in blood volume in the patient's head (e.g., brain).

To determine the OPVR percentage value, the OPVR evaluation module 436 generates a pulse waveform based on the PPG waveform in the PPG data (for example, by subtracting the baseline PPG signal as shown in FIG. 6-1), calculates a pulse volume for each pulse in the pulse waveform (for example, by integrating the area under the curve for each pulse), and generates a pulse volume waveform based on the pulse volume calculated for each pulse. The OPVR evaluation module 436 further normalizes the pulse volume waveform by dividing the pulse volume waveform with a maximum pulse volume value in the first phase and determine the percentage reduction in pulse volume based on a normalized maximum pulse volume value in the first phase and a normalized minimum pulse volume value in the third phase. OPVR evaluation module 436 generates OPVR data based on the determined OPVR percentage value.

In some implementations, the PPG evaluation unit 430 includes a postural orthostatic tachycardia pulse volume reduction (POT) evaluation module 438. POT evaluation module 438 determines the POT value which indicates an increase in heart rate upon standing—this increase is a symptom of postural orthostatic tachycardia syndrome.

The POT evaluation module 438 can correlate PPG data with the posture changes for determining the POT value. For example, in some implementations, the POT evaluation module 438 analyzes the PPG waveform within the PPG data to derive the heart rate waveform and determine the increased heart rate (e.g., maximum difference in heart rate) based on a heart rate value (e.g., minimum heart rate) in the first phase and a heart rate value (e.g., maximum heart rate) in the third phase. POT evaluation module 438 generates POT data based on the determined POT value.

In some implementations, the PPG evaluation unit 430 includes a first orthostatic hypovolemia (OHV1) evaluation module 440. The OHV1 evaluation module 440 determines drop in blood volume upon standing or sitting upright, based on the PPG data and posture data. The OHV1 value serves as an indicator of the drop in blood volume in the patient's head (e.g., brain).

To determine the OHV1 value, the OHV1 evaluation module 440 determines a determine a blood volume value (e.g., minimum blood volume value) in the first phase and a blood volume value (e.g., minimum blood volume value) in the third phase based on the waveform in the PPG data and posture data. The OHV1 evaluation module 440 further determined the difference between the blood volume value (e.g., minimum blood volume value) in the first phase and the blood volume (e.g., minimum blood volume value) in the third phase to determine OHV1 value. OHV1 evaluation module 440 generates OHV1 data based on the determined OHV1 value.

In some implementations, the PPG evaluation unit 430 includes a second orthostatic hypovolemia (OHV2) evaluation module 442. The OHV2 evaluation module 442 determines sustained reduction in blood volume based on the PPG data and posture data.

To determine the OHV2 value, the OHV2 evaluation module 442 determines a time the patient reaches a stabilized condition (e.g., in a condition recovered from OH) in the third phase based on one or more biometrics (e.g., blood volume, heart rate, difference amplitude, interbeat interval, pulse volume).

In some implementations, the OHV2 evaluation module 442 analyzes PPG waveform in the PPG data corresponding to (e.g., associated with) the third phase and determine a time when the PPG waveform becomes stabilized (e.g., blood volume fluctuates within a predetermined % value such as 10%). In some implementations, the OHV2 evaluation module 442 analyzes PPG waveform in the PPG data corresponding to (e.g., associated with) the third phase and determine a time when the heart rate derived from the PPG waveform becomes stabilized (e.g., heart rate fluctuates within a predetermined % value such as 10%). In some implementations, the OHV2 evaluation module 442 analyzes PPG waveform in the PPG data corresponding to (e.g., associated with) the third phase and determine a time when the pulse amplitude derived from the PPG waveform becomes stabilized (e.g., heart rate fluctuates within a predetermined % value such as 10%). In some implementations, the OHV2 evaluation module 442 analyzes PPG waveform in the PPG data corresponding to (e.g., associated with) the third phase and determine a time when the difference amplitude derived from the PPG waveform becomes stabilized (e.g., difference amplitude fluctuates within a predetermined % value such as 10%). In some implementations, the OHV2 evaluation module 442 analyzes PPG waveform in the PPG data corresponding to (e.g., associated with) the third phase and determine a time when the interbeat interval derived from the PPG waveform becomes stabilized (e.g., interbeat interval fluctuates within a predetermined % value such as 10%). In some implementations, the OHV2 evaluation module 442 analyzes PPG waveform in the PPG data corresponding to (e.g., associated with) the third phase and determine a time when the pulse volume derived from the PPG waveform becomes stabilized (e.g., pulse volume fluctuates within a predetermined % value such as 10%).

The OHV2 evaluation module 442 determines the blood volume value (e.g., minimum blood volume value) during the first phase from the PPG data and the blood volume value (e.g., minimum blood volume value) after the patient reaches a stabilized condition. The OHV2 evaluation module 442 further calculates the difference between the determined blood volume value in the first phase and the determined blood volume value after the patient is in a stabilized condition in the third phase to determine the OHV2 value. The OHV2 evaluation module 442 then generates OHV2 data based on the calculated OHV2 value.

In some implementations, the PPG evaluation unit 430 provides one or more results (e.g., orthostatic metrics such as POT value, OTC value, OHV1 value, OHV2 value, OPVR % value) to the healthcare provider through any suitable method. For example, the server may store the result in its storage, allowing the healthcare provider to access it. In some implementations, the server stores the result directly in the healthcare provider's computing device's storage. Alternatively, the server can store the result in the patient's electronic health record, enabling the healthcare provider to access it.

In some implementations, the PPG evaluation unit 430 provide the PPG data (e.g., PPG waveform) and/or other data derived from the PPG data such as monitoring data (e.g., heart rate waveform, pulse waveform, pulse volume waveform, pulse amplitude waveform, difference amplitude waveform) to the healthcare provider in any suitable method.

For example, in some implementations, the server stores the data in its storage, allowing the healthcare provider to access it. In some implementations, the server stores the data directly in the healthcare provider's computing device's storage. In some implementations, the server can store the data in the patient's electronic health record, enabling the healthcare provider to access the results.

In some implementations, the server includes orthostatic disorder evaluation unit 450 configured to determine whether the patient is more likely to having the orthostatic disorder based on one or more results (e.g., orthostatic metrics such as POT value, OTC value, OHV1 value, OHV2 value, OPVR % value) from the PPG evaluation unit 430.

For example, in some implementations, if the orthostatic disorder evaluation unit 450 determines that the OTC value from the PPG evaluation unit is equal to or greater than a predetermined threshold, it notifies the healthcare provider that the patient is more likely to have orthostatic hypotension (OH). Similarly, if the POT value is equal to or less than a predetermined threshold, the orthostatic disorder evaluation unit 450 alerts the healthcare provider of the patient's increased likelihood of having dysautonomia or impaired autonomic response. Additionally, if the OHV1 or OHV2 values meet or exceed their respective thresholds, the orthostatic disorder evaluation unit notifies the healthcare provider of the patient's likelihood of having the initial or sustained orthostatic hypotension, respectively. The notification can be provided in any suitable manner, such as through a message on the display. Additionally, in some cases, multiple metrics may be used to make a diagnosis. For example, if OHV1 is larger and OHV2 is smaller than a predetermined threshold, the system may inform the patient of initial orthostatic hypotension (IOH). Another example is if OHV1 is smaller and POT is larger than their respective thresholds, the system may diagnose postural orthostatic tachycardia syndrome (POTS).

In some implementations, the server provides the notification to the healthcare provider, such as through an on-screen notification on the healthcare provider's smartphone or email to the healthcare provider.

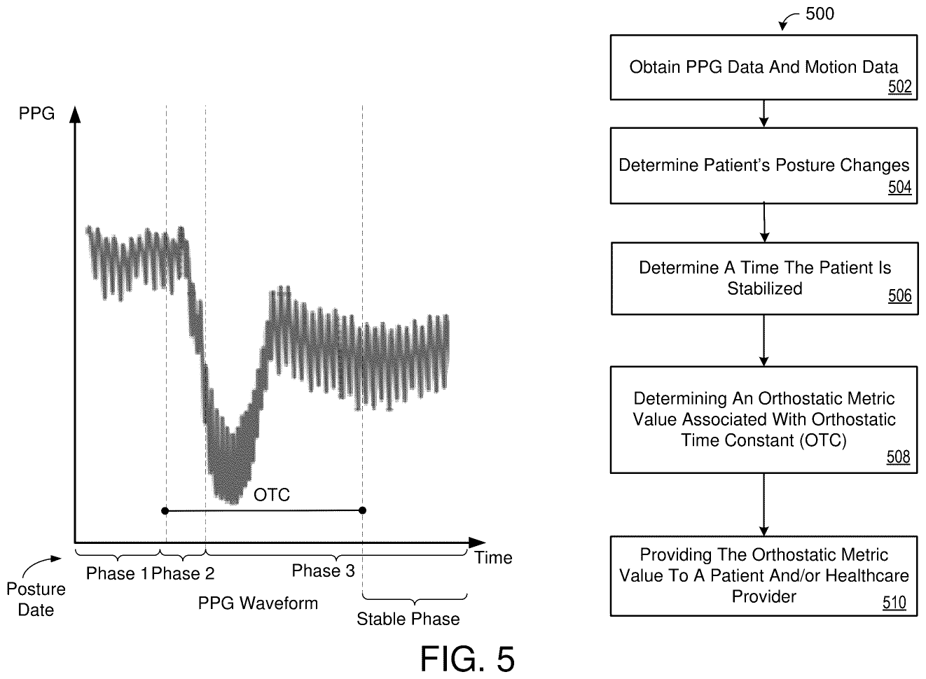

FIG. 5 includes a flowchart of an example arrangement of operations for a method for determining an orthostatic metric value associated with orthostatic time constant (OTC) in accordance with some implementations of this disclosure.

The method 500 may be performed by a computing device that may include hardware (circuitry, dedicated logic, data processing hardware etc.), software (such as is run on a general purpose computer system or a dedicated machine) one memory hardware, or a combination of both, which computing device may be included in any computer system or device. For simplicity of explanation, methods described herein are depicted and described as a series of acts. However, acts in accordance with this disclosure may occur in various orders and/or concurrently, and with other acts not presented and described herein. Further, not all illustrated acts may be used to implement the methods in accordance with the disclosed subject matter. In addition, those skilled in the art will understand and appreciate that the methods may alternatively be represented as a series of interrelated states via a state diagram or events. Additionally, the methods disclosed in this specification are capable of being stored on an article of manufacture, such as a non-transitory computer-readable medium, to facilitate transporting and transferring such methods to computing devices. The term article of manufacture, as used herein, is intended to encompass a computer program accessible from any computer-readable device or storage media. Although illustrated as discrete blocks, various blocks may be divided into additional blocks, combined into fewer blocks, or eliminated, depending on the desired implementation.

The orthostatic time constant (OTC) is a metric used to assess a patient's response to posture changes, particularly transitions from lying down or sitting to standing. It reflects how quickly the cardiovascular system stabilizes after such transitions, providing insight into blood volume shifts and heart rate regulation.

In the context of orthostatic metrics, the OTC value measures the time taken for the patient's blood volume or related physiological signals (e.g., pulse volume) to stabilize after standing up. This stabilization is typically indicated by the PPG waveform fluctuating within a certain percentage (e.g., 10%) of a baseline value. A shorter OTC suggests a faster stabilization, while a longer OTC may indicate a slower or impaired response, potentially associated with orthostatic disorder (e.g., orthostatic hypotension).

Thus, the OTC value helps assess the body's ability to adapt to sudden posture changes and serves as an important diagnostic tool for identifying issues like the orthostatic disorder, where blood volume drops or cardiovascular regulation is impaired upon standing.

The method 500, at operations 502, includes obtaining patient data including PPG data (e.g., PPG waveform) and motion data and/or posture data from a patient monitor. As described above, a PPG sensor of the patient monitor generates the PPG data (blood volume data) from an upper extremity body part of the patient. In this example, the upper extremity body of the patient is a patient's earlobe. As described above, the patient monitor generates the motion data and/or posture data (e.g., head/torso angle, acceleration in X-axis, Y-axis, and Z-axis) of the patient.

The method 500, at operations 504, includes determining the patient's posture changes based on the motion data or the posture data. As described above, a posture evaluation unit determines a tilt angle of the patient's head in relation to the ground based the motion data. For example, when the patient's head is approximately at 0°, the posture evaluation unit determines that the patient is lying down. When the head is approximately at 90°, the posture evaluation unit determines that the patient is standing up. A change in head position from approximately 0° to 90° indicates that the patient is in the process of transitioning to a standing (or sitting upright) posture. In some implementations, the posture evaluation unit generates posture data, including the start time and end time for each posture duration: first phase when the patient is lying down, second phase while the patient is in the process of standing up (or sitting upright), and third phase when the patient is standing up.

The method 500, at operations 506, determining a time the patient is stabilized after standing up.

For example, the computing device (OTC evaluation module) determines a time the patient reaches a stabilized condition (e.g., in a condition recovered from OH) in the third phase based on one or more biometrics (e.g., blood volume, heart rate, difference amplitude, interbeat interval, pulse volume).

In some implementations, the OTC evaluation module analyzes PPG waveform in the PPG data corresponding to the third phase and determine a time when the PPG waveform becomes stabilized (e.g., blood volume fluctuates within a predetermined % value such as 10%). In some implementations, the OTC evaluation module analyzes PPG waveform in the PPG data corresponding to the third phase and determine a time when the heart rate derived from the PPG waveform becomes stabilized (e.g., heart rate fluctuates within a predetermined % value such as 10%). In some implementations, the OTC evaluation module analyzes PPG waveform in the PPG data corresponding to the third phase and determine a time when the pulse amplitude derived from the PPG waveform becomes stabilized (e.g., heart rate fluctuates within a predetermined % value such as 10%). In some implementations, the OTC evaluation module analyzes PPG waveform in the PPG data corresponding to the third phase and determine a time when the difference amplitude derived from the PPG waveform becomes stabilized (e.g., difference amplitude fluctuates within a predetermined % value such as 10%). In some implementations, the OTC evaluation module analyzes PPG waveform in the PPG data corresponding to the third phase and determine a time when the interbeat interval derived from the PPG waveform becomes stabilized (e.g., interbeat interval fluctuates within a predetermined % value such as 10%). In some implementations, the OTC evaluation module analyzes PPG waveform in the PPG data corresponding to the third phase and determine a time when the pulse volume derived from the PPG waveform becomes stabilized (e.g., pulse volume fluctuates within a predetermined % value such as 10%).

The method 500, at operations 508, determining an orthostatic metric value associated with orthostatic time constant (OTC) (“OTC value”). As described above, in some implementations, the OTC evaluation module determine OTC value based on the time between the start time of the second phase and the determined time the patient is stabilized. In some implementations, the OTC evaluation module determine OTC value based on the time between the start time of the third phase and the determined time the patient is stabilized.

The method 500, at operations 510, providing the OTC value to the patient and/or healthcare provider. For example, the OTC value can be saved to the patient's electrical health record.

In some implementations, the method 500 includes providing a numeric score designed to quantitatively reflect the patient's orthostatic health based on the OTC value and other metrics. This scoring system may assign specific values to different health statuses, such as a score of 10 representing superior orthostatic health, a score of 5 indicating average orthostatic health, and a score of 1 denoting poor orthostatic health. This numeric representation allows for a clear and objective assessment of the patient's condition, making it easier for healthcare providers to understand the severity of the patient's orthostatic issues at a glance. By utilizing this scoring system, healthcare professionals can make more informed decisions regarding patient care and intervention strategies.

In addition to, or as an alternative to, the numeric score, the method 500 includes providing a visual representation of the patient's orthostatic health based on the OTC value and other metrics. This could involve a color-coded system that assigns specific colors to various levels of orthostatic health, such as green for good orthostatic health, yellow for average orthostatic health, and red for poor orthostatic health. This visual cue enhances the accessibility of the information, allowing both healthcare providers and patients to quickly interpret the data without needing to delve into the specifics of the numeric scores. Such visual representations serve as effective communication tools, facilitating discussions between patients and healthcare providers about the implications of the patient's orthostatic health and potential next steps in management or treatment.

FIG. 6-1 and FIG. 6-2 include a flowchart of an example arrangement of operations for a method for determining an orthostatic metric value associated with an orthostatic pulse volume reduction (OPVR) in accordance with some implementations of this disclosure.

The method 600 may be performed by a computing device that may include hardware (circuitry, dedicated logic, data processing hardware etc.), software (such as is run on a general purpose computer system or a dedicated machine) one memory hardware, or a combination of both, which computing device may be included in any computer system or device. For simplicity of explanation, methods described herein are depicted and described as a series of acts. However, acts in accordance with this disclosure may occur in various orders and/or concurrently, and with other acts not presented and described herein. Further, not all illustrated acts may be used to implement the methods in accordance with the disclosed subject matter. In addition, those skilled in the art will understand and appreciate that the methods may alternatively be represented as a series of interrelated states via a state diagram or events. Additionally, the methods disclosed in this specification are capable of being stored on an article of manufacture, such as a non-transitory computer-readable medium, to facilitate transporting and transferring such methods to computing devices. The term article of manufacture, as used herein, is intended to encompass a computer program accessible from any computer-readable device or storage media. Although illustrated as discrete blocks, various blocks may be divided into additional blocks, combined into fewer blocks, or eliminated, depending on the desired implementation.

In this example, orthostatic pulse volume reduction (OPVR) is a metric used to assess how the blood volume in a person's body changes when they transition from a lying or sitting position to standing. Specifically, OPVR measures the reduction in blood volume reaching the head (e.g., brain) during this postural change.

This reduction occurs due to the effects of gravity, which causes blood to pool in the lower extremities when a person stands up, potentially leading to a temporary drop in blood supply to the brain.

In this example, the OPVR value is determined using photoplethysmography (PPG) data by deriving the pulse volume on earlobe, which reflects the amount of blood flowing through the vessels at the earlobe. When a person stands up, the pulse volume decreases as blood shifts away from the upper body. The OPVR evaluation module calculates the percentage drop in pulse volume to indicate the extent of this reduction.

A higher OPVR percentage suggests a significant reduction in blood volume upon standing, which can be an indicator of orthostatic intolerance or conditions like orthostatic hypotension (OH), where the body has difficulty maintaining adequate blood flow and blood pressure when changing posture.

The method 600, at operations 602, includes obtaining patient data including PPG data (e.g., PPG waveform) and motion data and/or posture data from a patient monitor. As described above, a PPG sensor of the patient monitor generates the PPG data (blood volume data) from an upper extremity body part of the patient. In this example, the upper extremity body of the patient is a patient's earlobe. As described above, the patient monitor generates the motion data (e.g., head/torso angle, acceleration in X-axis, Y-axis, and Z-axis) of the patient.

The method 600, at operations 604, includes determining the patient's posture changes based on the motion data or the posture data. As described above, a posture evaluation unit determine tilt angle of the patient's head in relation to the ground based the motion data. For example, when the patient's head is approximately at 0°, the posture evaluation unit determines that the patient is lying down. When the head is approximately at 90°, the posture evaluation unit determines that the patient is standing up. A change in head position from approximately 0° to 90° indicates that the patient is in the process of transitioning to a standing (or sitting upright) posture. In some implementations, the posture evaluation unit generates posture data, including the start time and end time for each posture duration: first phase when the patient is lying down, second phase while the patient is in the process of standing up (or sitting upright), and third phase when the patient is standing up.

The method 600, at operations 606, includes generating a pulse waveform based the PPG waveform. The PPG waveform represents blood volume changes in microvascular tissue. It typically consists of a pulsatile (AC) component, corresponding to the heartbeat, and a steady (DC) component, related to non-pulsatile blood volume. In some implementations, OPVR evaluation module generates the pulse waveform by removing the DC component (baseline) from the PPG waveform to focus on the pulsatile component that reflect the changes in blood volume.

The method 600, at operation 608, includes generating a pulse volume waveform based on the pulse waveform. The OPVR evaluation module determines the pulse volume (PV) by calculating the integrated area for each pulse and generates a corresponding pulse volume waveform.

The method 600, at operation 610, includes generating a normalized pulse volume waveform based on the pulse volume waveform. The OPVR evaluation module generates the normalized pulse volume waveform by dividing the pulse volume waveform with a maximum pulse volume value in the first phase.

The method 600, at operation 612, includes determining the orthostatic metric value associated with OPVR (e.g., percentage reduction in pulse volume) based on a normalized maximum pulse volume value (in the first phase) and a normalized minimum pulse volume value (in the third phase).

The method 600, at operations 614, providing the OPVR percentage value to the patient and/or healthcare provider. For example, the OPVR percentage value can be saved to the patient's electrical health record.

In some implementations, the method 600 includes providing a numeric score designed to quantitatively reflect the patient's orthostatic health based on the OPVR percentage value and other metrics. This scoring system may assign specific values to different health statuses, such as a score of 10 representing superior orthostatic health, a score of 5 indicating average orthostatic health, and a score of 1 denoting poor orthostatic health. This numeric representation allows for a clear and objective assessment of the patient's condition, making it easier for healthcare providers to understand the severity of the patient's orthostatic issues at a glance. By utilizing this scoring system, healthcare professionals can make more informed decisions regarding patient care and intervention strategies.

In addition to, or as an alternative to, the numeric score, the method 600 includes providing a visual representation of the patient's orthostatic health based on the OPVR percentage value and other metrics. This could involve a color-coded system that assigns specific colors to various levels of orthostatic health, such as green for good orthostatic health, yellow for average orthostatic health, and red for poor orthostatic health. This visual cue enhances the accessibility of the information, allowing both healthcare providers and patients to quickly interpret the data without needing to delve into the specifics of the numeric scores. Such visual representations serve as effective communication tools, facilitating discussions between patients and healthcare providers about the implications of the patient's orthostatic health and potential next steps in management or treatment.

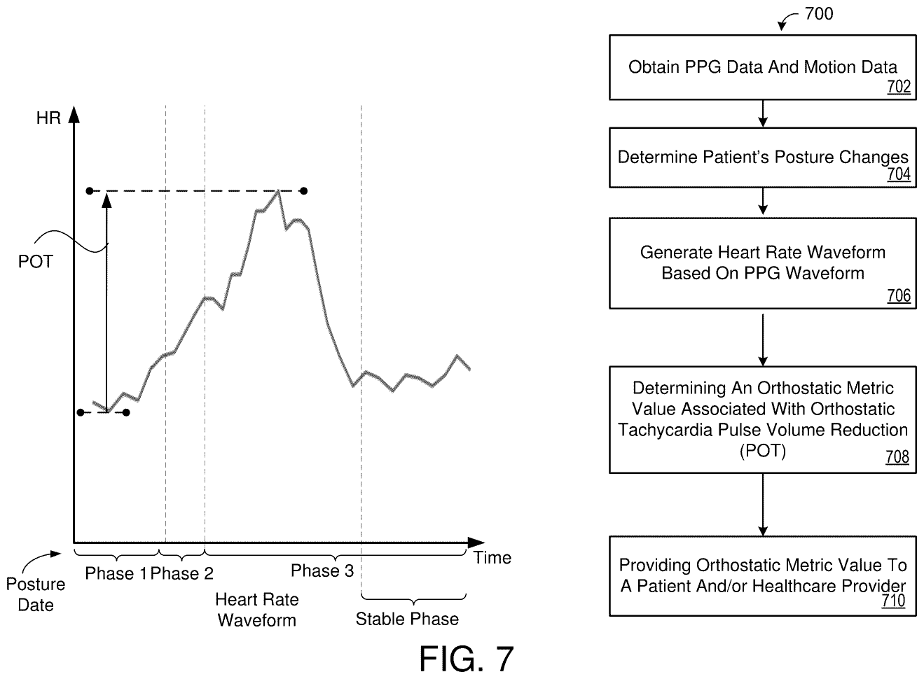

FIG. 7 includes a flowchart of an example arrangement of operations for a method for determining an orthostatic metric value associated with postural orthostatic tachycardia pulse volume reduction (POT) in accordance with some implementations of this disclosure.

The method 700 may be performed by a computing device that may include hardware (circuitry, dedicated logic, data processing hardware etc.), software (such as is run on a general purpose computer system or a dedicated machine) one memory hardware, or a combination of both, which computing device may be included in any computer system or device. For simplicity of explanation, methods described herein are depicted and described as a series of acts. However, acts in accordance with this disclosure may occur in various orders and/or concurrently, and with other acts not presented and described herein. Further, not all illustrated acts may be used to implement the methods in accordance with the disclosed subject matter. In addition, those skilled in the art will understand and appreciate that the methods may alternatively be represented as a series of interrelated states via a state diagram or events. Additionally, the methods disclosed in this specification are capable of being stored on an article of manufacture, such as a non-transitory computer-readable medium, to facilitate transporting and transferring such methods to computing devices. The term article of manufacture, as used herein, is intended to encompass a computer program accessible from any computer-readable device or storage media. Although illustrated as discrete blocks, various blocks may be divided into additional blocks, combined into fewer blocks, or eliminated, depending on the desired implementation.

Postural Orthostatic Tachycardia (POT) refers to a condition characterized by a significant increase in heart rate when a person transitions from lying down to standing or sitting. In this example, the Postural Orthostatic Tachycardia pulse volume reduction (POT) metric captures this physiological response by assessing the change in heart rate during the posture change.

During the posture change, the heart compensates for a drop in blood volume (due to gravity pulling blood toward the lower extremities) by significantly increasing the heart rate. A low POT value in a patient indicates a slow response to the drop in blood volume, suggesting the patient is more likely to have orthostatic hypotension (OH). This metric helps in diagnosing and understanding the severity of the condition, as well as how the body responds to posture changes, particularly in maintaining adequate blood circulation.

The method 700, at operations 702, includes obtaining patient data including PPG data (e.g., PPG waveform) and motion data and/or posture data from a patient monitor associated with a patient's computing device. As described above, a PPG sensor of the patient monitor generates the PPG data (blood volume data) from an upper extremity body part of the patient. In this example, the upper extremity body of the patient is a patient's earlobe. As described above, a motion sensor of the patient monitor generates the motion data and/or posture data (e.g., head/torso angle, acceleration in X-axis, Y-axis, and Z-axis) of the patient.

The method 700, at operations 704, includes determining the patient's posture changes based on the motion data or the posture data. As described above, a posture evaluation unit determines a tilt angle of the patient's head in relation to the ground based the motion data. For example, when the patient's head is approximately at 0°, the posture evaluation unit determines that the patient is lying down. When the head is approximately at 90°, the posture evaluation unit determines that the patient is standing up. A change in head position from approximately 0° to 90° indicates that the patient is in the process of transitioning to a standing (or sitting upright) posture. In some implementations, the posture evaluation unit generates posture data, including the start time and end time for each posture duration: first phase when the patient is lying down, second phase while the patient is in the process of standing up (or sitting upright), and third phase when the patient is standing up.

The method 700, at operation 706, includes generating a heart rate waveform based on the PPG waveform from the PPG data. In some implementations, the POT evaluation module generates the heart rate waveform by detecting peaks in the PPG waveform and calculating the time between consecutive peaks. These intervals are used to derive heart rate values, which are then used to create the heart rate waveform.

The method 700, at operation 708, includes determining the orthostatic metric value associated with Postural Orthostatic Tachycardia (POT) by calculating the increase in heart rate (e.g., the maximum difference in heart rate). This is determined by comparing the heart rate value (e.g., minimum heart rate) in the first phase to the heart rate value (e.g., maximum heart rate) in the third phase.

The method 700, at operations 710, providing the POT value to the patient and/or healthcare provider. For example, the POT value can be saved to the patient's electrical health record.

In some implementations, the method 700 includes providing a numeric score designed to quantitatively reflect the patient's orthostatic health based on the POT value and other metrics. This scoring system may assign specific values to different health statuses, such as a score of 10 representing superior orthostatic health, a score of 5 indicating average orthostatic health, and a score of 1 denoting poor orthostatic health. This numeric representation allows for a clear and objective assessment of the patient's condition, making it easier for healthcare providers to understand the severity of the patient's orthostatic issues at a glance. By utilizing this scoring system, healthcare professionals can make more informed decisions regarding patient care and intervention strategies.

In addition to, or as an alternative to, the numeric score, the method 700 includes providing a visual representation of the patient's orthostatic health based on the POT value and other metrics. This could involve a color-coded system that assigns specific colors to various levels of orthostatic health, such as green for good orthostatic health, yellow for average orthostatic health, and red for poor orthostatic health. This visual cue enhances the accessibility of the information, allowing both healthcare providers and patients to quickly interpret the data without needing to delve into the specifics of the numeric scores. Such visual representations serve as effective communication tools, facilitating discussions between patients and healthcare providers about the implications of the patient's orthostatic health and potential next steps in management or treatment.

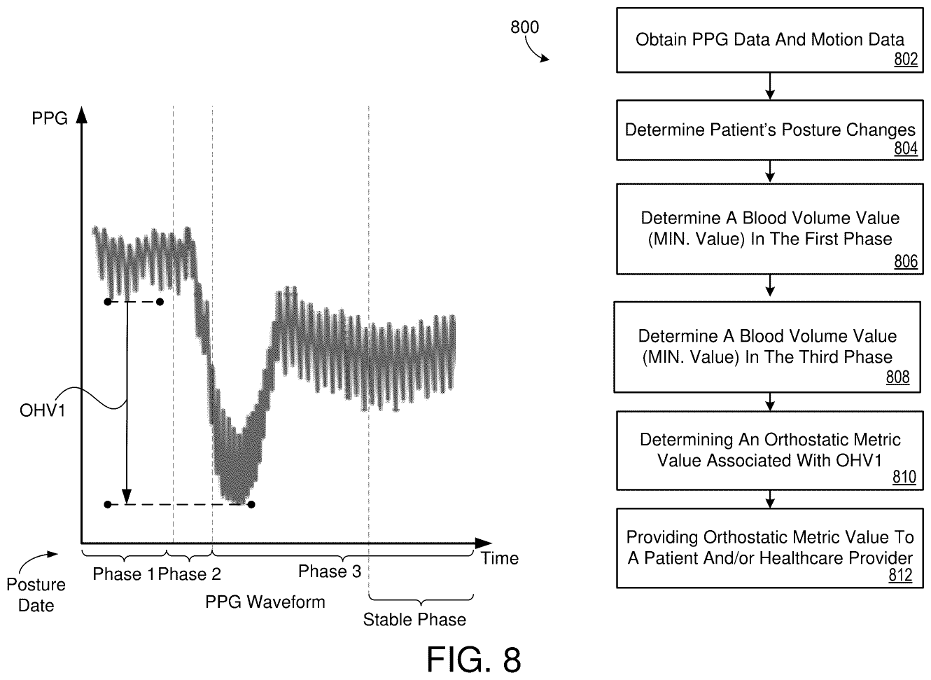

FIG. 8 include a flowchart of an example arrangement of operations for a method for determining an orthostatic metric value associated with orthostatic hypovolemia (OHV1) in accordance with some implementations of this disclosure.

The method 800 may be performed by a computing device that may include hardware (circuitry, dedicated logic, data processing hardware etc.), software (such as is run on a general purpose computer system or a dedicated machine) one memory hardware, or a combination of both, which computing device may be included in any computer system or device. For simplicity of explanation, methods described herein are depicted and described as a series of acts. However, acts in accordance with this disclosure may occur in various orders and/or concurrently, and with other acts not presented and described herein. Further, not all illustrated acts may be used to implement the methods in accordance with the disclosed subject matter. In addition, those skilled in the art will understand and appreciate that the methods may alternatively be represented as a series of interrelated states via a state diagram or events. Additionally, the methods disclosed in this specification are capable of being stored on an article of manufacture, such as a non-transitory computer-readable medium, to facilitate transporting and transferring such methods to computing devices. The term article of manufacture, as used herein, is intended to encompass a computer program accessible from any computer-readable device or storage media. Although illustrated as discrete blocks, various blocks may be divided into additional blocks, combined into fewer blocks, or eliminated, depending on the desired implementation.

As described above, the first orthostatic hypovolemia (OHV1) refers to a temporary drop in blood volume that occurs when a person stands up from a lying or sitting position. This condition can result from the pooling of blood in the lower extremities due to gravity, which reduces the amount of blood returning to the heart. The body typically compensates for this by constricting blood vessels and increasing heart rate to maintain blood pressure and circulation.

The OHV1 metric specifically represents this initial, short-term blood volume reduction upon standing. In this example, the OHV1 evaluation module measures the blood volume reduction using data from photoplethysmography (PPG) sensors, which detect changes in blood flow. The module calculates the OHV1 value by determining the immediate drop in pulse volume upon standing, capturing the transient hypovolemia before the body stabilizes.

A higher OHV1 value suggests a more pronounced drop in blood volume, which may indicate impaired compensatory mechanisms and could be a sign of orthostatic hypotension (OH) or other circulatory dysfunctions. The OHV1 metric helps in assessing the body's ability to regulate blood volume and maintain proper circulation during posture changes.

The method 800, at operations 802, includes obtaining patient data including PPG data (e.g., PPG waveform) and motion data and/or posture data from a patient monitor. As described above, a PPG sensor of the patient monitor generates the PPG data (blood volume data) from an upper extremity body part of the patient. In this example, the upper extremity body of the patient is a patient's earlobe. As described above, the patient monitor generates the motion data and/or posture data (e.g., head/torso angle, acceleration in X-axis, Y-axis, and Z-axis) of the patient.

The method 800, at operations 804, includes determining the patient's posture changes based on the motion data. As described above, a posture evaluation unit determines a tilt angle of the patient's head in relation to the ground based the motion data. For example, when the patient's head is approximately at 0°, the posture evaluation unit determines that the patient is lying down. When the head is approximately at 90°, the posture evaluation unit determines that the patient is standing up. A change in head position from approximately 0° to 90° indicates that the patient is in the process of transitioning to a standing (or sitting upright) posture. In some implementations, the posture evaluation unit generates posture data, including the start time and end time for each posture duration: first phase when the patient is lying down, second phase while the patient is in the process of standing up (or sitting upright), and third phase when the patient is standing up.

The method 800, at operations 806, includes determining a blood volume value (minimum blood volume value) in the first phase based on the PPG data.

The method 800, at operations 808, includes determining a blood volume value (minimum blood volume value) in the third phase based on the PPG data.

The method 800, at operations 810, includes determining a difference between the blood volume value (e.g., minimum blood volume value) in the first phase and the blood volume (e.g., minimum blood volume value) in the third phase to determine the orthostatic metric value associated with orthostatic hypovolemia (OHV1).

The method 800, at operations 812, includes providing the OHV1 value to the patient and/or healthcare provider. For example, the OHV1 value can be saved to the patient's electrical health record. It will be appreciated that the OHV1 value may be used, at least in part, to diagnose a disorder by comparing the OHV1 value with a normal OHV1 value (which may be collected from patient data stored in a patient database, for example).

In some implementations, the method 800 includes providing a numeric score designed to quantitatively reflect the patient's orthostatic health based on the OHV1 value and other metrics. This scoring system may assign specific values to different health statuses, such as a score of 10 representing superior orthostatic health, a score of 5 indicating average orthostatic health, and a score of 1 denoting poor orthostatic health. This numeric representation allows for a clear and objective assessment of the patient's condition, making it easier for healthcare providers to understand the severity of the patient's orthostatic issues at a glance. By utilizing this scoring system, healthcare professionals can make more informed decisions regarding patient care and intervention strategies.

In addition to, or as an alternative to, the numeric score, the method 800 includes providing a visual representation of the patient's orthostatic health based on the OHV1 value and other metrics. This could involve a color-coded system that assigns specific colors to various levels of orthostatic health, such as green for good orthostatic health, yellow for average orthostatic health, and red for poor orthostatic health. This visual cue enhances the accessibility of the information, allowing both healthcare providers and patients to quickly interpret the data without needing to delve into the specifics of the numeric scores. Such visual representations serve as effective communication tools, facilitating discussions between patients and healthcare providers about the implications of the patient's orthostatic health and potential next steps in management or treatment.

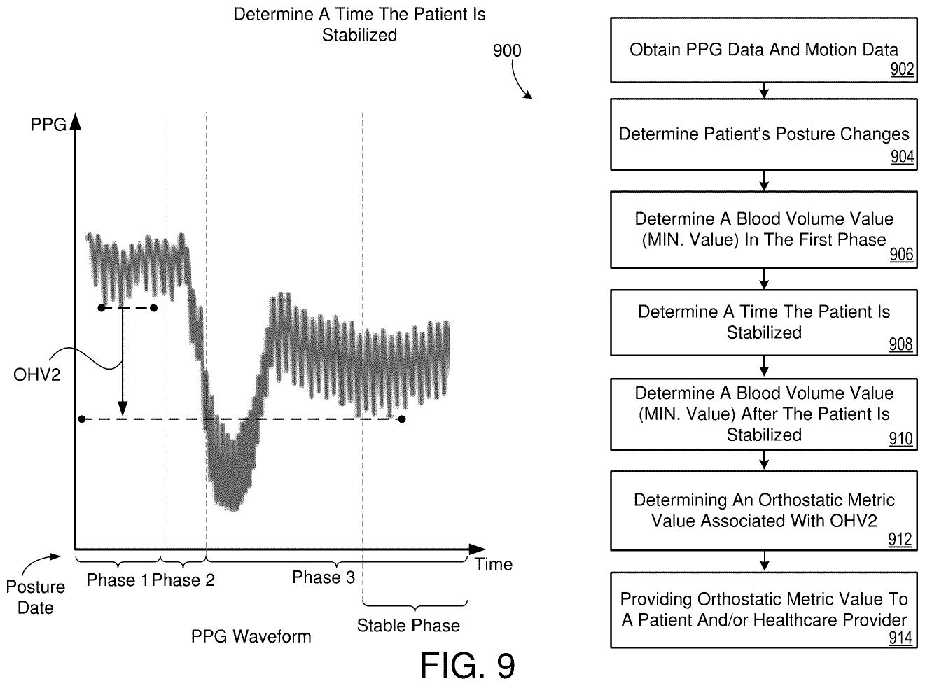

FIG. 9 include a flowchart of an example arrangement of operations for a method for determining an orthostatic metric value associated with orthostatic hypovolemia (OHV2) in accordance with some implementations of this disclosure.

The method 900 may be performed by a computing device that may include hardware (circuitry, dedicated logic, data processing hardware etc.), software (such as is run on a general purpose computer system or a dedicated machine) one memory hardware, or a combination of both, which computing device may be included in any computer system or device. For simplicity of explanation, methods described herein are depicted and described as a series of acts. However, acts in accordance with this disclosure may occur in various orders and/or concurrently, and with other acts not presented and described herein. Further, not all illustrated acts may be used to implement the methods in accordance with the disclosed subject matter. In addition, those skilled in the art will understand and appreciate that the methods may alternatively be represented as a series of interrelated states via a state diagram or events. Additionally, the methods disclosed in this specification are capable of being stored on an article of manufacture, such as a non-transitory computer-readable medium, to facilitate transporting and transferring such methods to computing devices. The term article of manufacture, as used herein, is intended to encompass a computer program accessible from any computer-readable device or storage media. Although illustrated as discrete blocks, various blocks may be divided into additional blocks, combined into fewer blocks, or eliminated, depending on the desired implementation.

As described above, orthostatic hypovolemia (OHV2) refers to the assessment of blood volume changes that occur after a person stands from a lying or sitting position, focusing on the body's stabilization phase. Unlike OHV1, which measures the immediate drop in blood volume upon standing, OHV2 evaluates the blood volume after the body has adjusted to the upright position.

In this example, the OHV2 evaluation module calculates the OHV2 value by comparing (e.g., the difference between) the minimum blood volume recorded in the first phase to the minimum blood volume measured once the body reaches a stabilized condition (within the third phase). A higher OHV2 value indicates a greater discrepancy between these measurements, suggesting potential difficulties in the body's ability to regulate blood volume during posture changes.

This metric facilitates diagnosing conditions such as orthostatic hypotension (OH) and provides insights into cardiovascular health, helping healthcare professionals evaluate the efficacy of compensatory mechanisms in response to changes in posture.

The method 900, at operations 902, includes obtaining patient data including PPG data (e.g., PPG waveform) and motion data and/or posture data from a patient monitor. As described above, a PPG sensor of the patient monitor generates the PPG data (blood volume data) from an upper extremity body part of the patient. In this example, the upper extremity body of the patient is a patient's earlobe. As described above, the patient monitor generates the motion data and/or posture data (e.g., head/torso angle acceleration in X-axis, Y-axis, and Z-axis) of the patient.

The method 900, at operations 904, includes determining the patient's posture changes based on the motion data. As described above, a posture evaluation unit determine a tilt angle of the patient's head in relation to the ground based the motion data. For example, when the patient's head is approximately at 0°, the posture evaluation unit determines that the patient is lying down. When the head is approximately at 90°, the posture evaluation unit determines that the patient is standing up. A change in head position from approximately 0° to 90° indicates that the patient is in the process of transitioning to a standing (or sitting upright) posture. In some implementations, the posture evaluation unit generates posture data, including the start time and end time for each posture duration: first phase when the patient is lying down, second phase while the patient is in the process of standing up (or sitting upright), and third phase when the patient is standing up.

The method 900, at operations 906, includes determining a blood volume value (minimum blood volume value) in the first phase based on the PPG data.

The method 900, at operations 908, determining a time the patient is stabilized after standing up.

For example, the computing device (OHV2 evaluation module) determines a time the patient reaches a stabilized condition (e.g., in a condition recovered from OH) in the third phase based on one or more biometrics (e.g., blood volume, heart rate, difference amplitude, interbeat interval, pulse volume).

In some implementations, the OHV2 evaluation module analyzes PPG waveform in the PPG data corresponding to the third phase and determine a time when the PPG waveform becomes stabilized (e.g., blood volume fluctuates within a predetermined % value such as 10%). In some implementations, the OHV2 evaluation module analyzes PPG waveform in the PPG data corresponding to the third phase and determine a time when the heart rate derived from the PPG waveform becomes stabilized (e.g., heart rate fluctuates within a predetermined % value such as 10%). In some implementations, the OHV2 evaluation module analyzes PPG waveform in the PPG data corresponding to the third phase and determine a time when the pulse amplitude derived from the PPG waveform becomes stabilized (e.g., heart rate fluctuates within a predetermined % value such as 10%). In some implementations, the OHV2 evaluation module analyzes PPG waveform in the PPG data corresponding to the third phase and determine a time when the difference amplitude derived from the PPG waveform becomes stabilized (e.g., difference amplitude fluctuates within a predetermined % value such as 10%). In some implementations, the OHV2 evaluation module analyzes PPG waveform in the PPG data corresponding to the third phase and determine a time when the interbeat interval derived from the PPG waveform becomes stabilized (e.g., interbeat interval fluctuates within a predetermined % value such as 10%). In some implementations, the OHV2 evaluation module analyzes PPG waveform in the PPG data corresponding to the third phase and determine a time when the pulse volume derived from the PPG waveform becomes stabilized (e.g., pulse volume fluctuates within a predetermined % value such as 10%).

The method 900, at operations 910, determining a blood volume value (minimum blood volume value) after the patient is stabilized in the first phase based on the PPG data.

The method 900, at operations 912, determining a blood volume value (minimum blood volume value) after the patient is stabilized in the first phase based on the PPG data.

The method 900, at operation 912, includes determining the difference between the blood volume value (e.g., minimum blood volume value) in the first phase and the blood volume value (e.g., minimum blood volume value) after the patient has stabilized in the third phase to determine the orthostatic metric value associated with orthostatic hypovolemia (OHV2).

The method 900, at operation 914, includes providing the OHV2 value to the patient and/or healthcare provider. For example, the OHV2 value can be saved to the patient's electronic health record.

In some implementations, the method 900 includes providing a numeric score designed to quantitatively reflect the patient's orthostatic health based on the OHV2 value and other metrics. This scoring system may assign specific values to different health statuses, such as a score of 10 representing superior orthostatic health, a score of 5 indicating average orthostatic health, and a score of 1 denoting poor orthostatic health. This numeric representation allows for a clear and objective assessment of the patient's condition, making it easier for healthcare providers to understand the severity of the patient's orthostatic issues at a glance. By utilizing this scoring system, healthcare professionals can make more informed decisions regarding patient care and intervention strategies.

In addition to, or as an alternative to, the numeric score, the method 900 includes providing a visual representation of the patient's orthostatic health based on the OHV2 value and other metrics. This could involve a color-coded system that assigns specific colors to various levels of orthostatic health, such as green for good orthostatic health, yellow for average orthostatic health, and red for poor orthostatic health. This visual cue enhances the accessibility of the information, allowing both healthcare providers and patients to quickly interpret the data without needing to delve into the specifics of the numeric scores. Such visual representations serve as effective communication tools, facilitating discussions between patients and healthcare providers about the implications of the patient's orthostatic health and potential next steps in management or treatment.

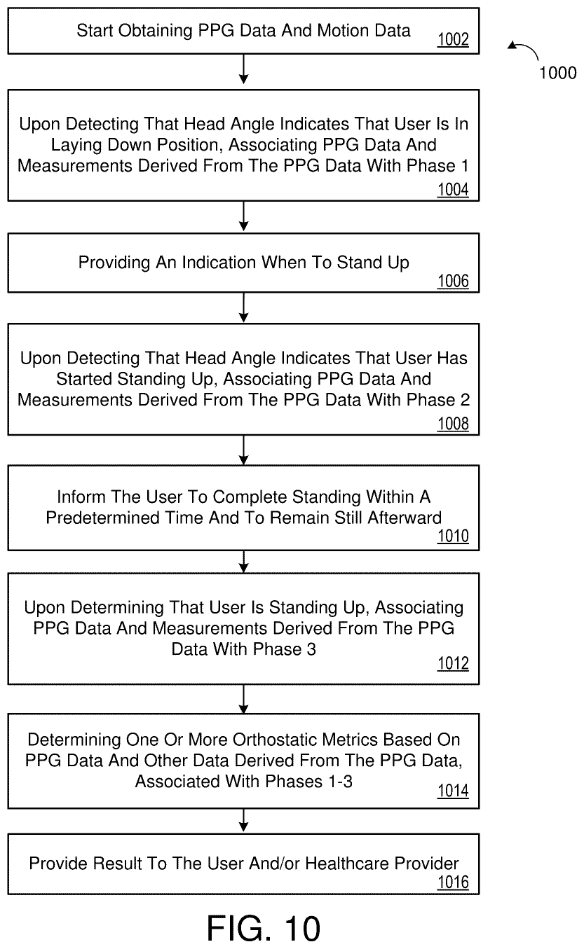

FIG. 10 includes a flowchart of an example arrangement of operations for a method for monitoring orthostatic metrics in a standup test in accordance with some implementations of this disclosure.

The method 1000 may be performed by a computing device that may include hardware (circuitry, dedicated logic, data processing hardware etc.), software (such as is run on a general purpose computer system or a dedicated machine) one memory hardware, or a combination of both, which computing device may be included in any computer system or device. For simplicity of explanation, methods described herein are depicted and described as a series of acts. However, acts in accordance with this disclosure may occur in various orders and/or concurrently, and with other acts not presented and described herein. Further, not all illustrated acts may be used to implement the methods in accordance with the disclosed subject matter. In addition, those skilled in the art will understand and appreciate that the methods may alternatively be represented as a series of interrelated states via a state diagram or events. Additionally, the methods disclosed in this specification are capable of being stored on an article of manufacture, such as a non-transitory computer-readable medium, to facilitate transporting and transferring such methods to computing devices. The term article of manufacture, as used herein, is intended to encompass a computer program accessible from any computer-readable device or storage media. Although illustrated as discrete blocks, various blocks may be divided into additional blocks, combined into fewer blocks, or eliminated, depending on the desired implementation.

The method 1000, at operation 1002, includes starting to obtain PPG data and motion data from a patient monitor. Based on the motion data (e.g., acceleration in X/Y/Z), the user's head angle is recorded. Additionally, other biometrics (e.g., heart rate waveform, pulse volume waveform, difference amplitude waveform, pulse amplitude waveform) are continuously derived from the PPG data.

The method 1000, at operation 1004, includes, upon detecting that the head/torso angle indicates the user is in a lying-down position, associating PPG data and other biometrics derived from the PPG data with Phase 1. For example, the start and end of Phase 1 are added to the timeline in the biometrics. In some implementations, based on the user's input that indicates that the user is in laying down position, the head/torso angle can be calibrated to zero.

The method 1000, at operation 1006, includes providing an indication to the user when to start standing up. The indication can be a countdown timer (e.g., displayed visually, through sound, or voice). Alternatively, the indication can be an alert, such as a beep sound or a flashing light. As a result, the user remains in the laying-down positon for at least 1 minute or greater (e.g., 3-5 minutes).

The method 1000, at operation 1008, includes, upon detecting that head angle (approximately 0° or angel between −15° and −30°) indicates that user has started standing up, associating PPG data and other biometrics derived from the PPG data with Phase 2. For example, the start and end of Phase 2 are added to the timeline in the biometrics.

The method 1000, at operation 1010, includes informing the user to complete standing within a predetermined time and to remain still afterward. As a result, the user remains fully standing for at least 1 minute or greater (e.g., 3-5 minutes).

The method 1000, at operation 1012, includes, when determining that the head/torso angle is between approximately 60° to 90° and the user in not in motion, indicating that the user is fully standing, associating PPG data and other biometrics derived from the PPG data with Phase 3. For example, the start and end of Phase 3 are added to the timeline in the biometrics. Using the motion data (e.g., acceleration in X-axis, Y-axis, and Z-axis), it is possible to determine whether the user is in motion. Additionally, this data can be analyzed to assess the angle of the head or torso, providing insights into the user's posture.

The method 900, at operation 1014, includes determining one or more orthostatic metrics (e.g., POT, OHV1, OHV2, OPVR, OTC) based on PPG data and other data derived from the PPG data, associated with Phases 1-3. In some implementations, the results can be sent to healthcare provider and/or caregiver. In some implementations, the results can be added to the user's electrical health record. In some implementations, the results can be upload to a cloud server.

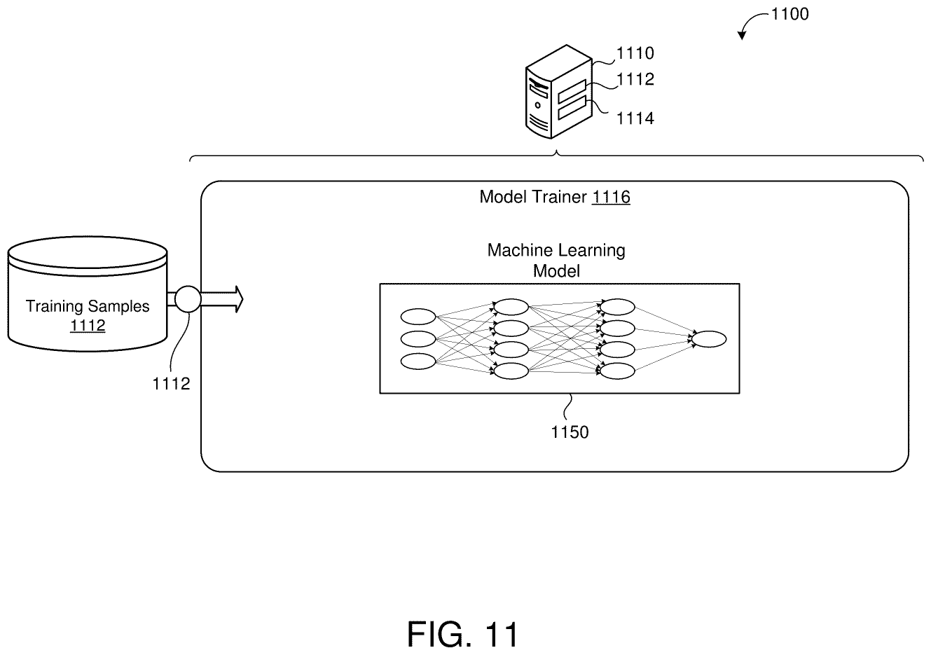

Referring to FIG. 11, in some implementations, an example system 1100, includes a processing system 1110. The processing system 1110 may be a single computer, multiple computers, or a distributed system (e.g., a cloud environment) having fixed or scalable/clastic computing resources 1112 (e.g., data processing hardware) and/or storage resources 1114 (e.g., memory hardware). The processing system 1100 executes a model trainer 1116. The model trainer 1116 trains a machine learning model 1150 (e.g., a deep neural network (DNN)) to make predictions based on input data. For example, the model trainer 1116 trains a convolutional neural network (CNN) of the machine learning model 1150. The model trainer 1116 trains the machine learning model 1150 on a set of training samples 1112. In some implementations, the set of training samples 1112 is stored in the storage resource 1114.

In some implementations, each training sample 1012 may include one or more orthostatic metric values (e.g., OTC, POT, OPVR, OHV1, OHV2), along with a corresponding label indicating whether the user associated with those orthostatic metric values has been diagnosed with orthostatic hypotension (OH) disorder. This labeling is crucial for supervised learning, where the model learns to recognize patterns and relationships in the data that correspond to specific outcomes. In some implementations, each training sample 1112 includes additional user information, such as medical history, weight, height, smoking status (e.g., smoker or non-smoker), drinking status (e.g., drinker or non-drinker), and family medical history.

The model trainer 1116 obtains the training samples 1112, in some implementations, from the storage resources 1114 or other devices suitable for storing the training samples. During the training phase, the model adjusts its internal parameters based on the errors in its predictions compared to the actual outcomes in the training data. This process continues iteratively, using techniques such as backpropagation and gradient descent, until the model achieves satisfactory performance metrics, such as accuracy and loss.

Once trained, the machine learning model 1150 can be deployed to analyze new patient data, enabling real-time predictions regarding the likelihood of orthostatic disorder based on the derived orthostatic metrics. For example, the machine learning model 1150 can be deploy to orthostatic disorder evaluation unit of the user's device, orthostatic disorder evaluation unit of the healthcare provider's device, and orthostatic disorder evaluation unit of the server—updating or adjusting predetermined thresholds or predetermined parameters associated with determining the orthostatic disorder. This predictive capability can assist healthcare providers in making informed decisions about patient management and treatment strategies.

In some implementations, a processing system 1110 includes any suitable model (e.g. AI model) in addition to the machine learning model 1150. In some implementations, a processing system 1110 includes any suitable model (e.g. AI model) in lieu of the machine learning model 1150.

In some implementations, the outputs generated by the machine learning model 1150, or any suitable alternative model, can manifest as classifications that indicate the orthostatic health status of a patient. These classifications may utilize a color-coded system—such as red, yellow, and green—to provide an immediate visual representation of the patient's condition. In some implementations, the model may produce a numeric score that quantitatively reflects the patient's orthostatic health. This dual approach, combining qualitative and quantitative metrics, enables healthcare providers to quickly assess patient status and make timely interventions when necessary, ultimately enhancing the quality of care provided.

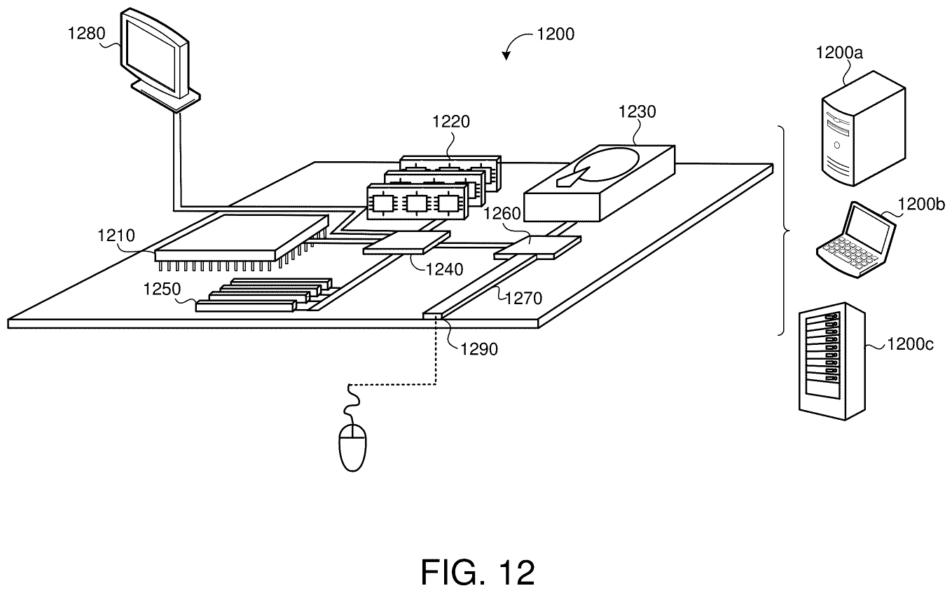

FIG. 12 includes schematic view of an example computing device 1200 that may be used to implement the systems and methods described in this document. The computing device 1200 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 1200 includes a processor 1210, memory 1220, a storage device 1230, a high-speed interface/controller 1240 connecting to the memory 1220 and high-speed expansion ports 1250, and a low speed interface/controller 1260 connecting to a low speed bus 1270 and a storage device 1230. Each of the components 1210, 1220, 1230, 1240, 1250, and 1260, are interconnected using various busses, and may be mounted on a common motherboard or in other manners as appropriate. The processor 1210 can process instructions for execution within the computing device 1200, including instructions stored in the memory 1220 or on the storage device 1230 to display graphical information for a graphical user interface (GUI) on an external input/output device, such as display 1280 coupled to high speed interface 1240. In other implementations, multiple processors and/or multiple buses may be used, as appropriate, along with multiple memories and types of memory. Also, multiple computing devices 1200 may be connected, with each device providing portions of the necessary operations (e.g., as a server bank, a group of blade servers, or a multi-processor system).

The memory 1220 stores information non-transitorily within the computing device 700. The memory 1220 may be a computer-readable medium, a volatile memory unit(s), or non-volatile memory unit(s). The non-transitory memory 1220 may be physical devices used to store programs (e.g., sequences of instructions) or data (e.g., program state information) on a temporary or permanent basis for use by the computing device 1200. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 1230 is capable of providing mass storage for the computing device 1200. In some implementations, the storage device 1230 is a computer-readable medium. In various different implementations, the storage device 1230 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 1220, the storage device 1230, or memory on processor 1210.

The high speed controller 1240 manages bandwidth-intensive operations for the computing device 1200, while the low speed controller 1260 manages lower bandwidth-intensive operations. Such allocation of duties is exemplary only. In some implementations, the high-speed controller 1240 is coupled to the memory 1220, the display 1280 (e.g., through a graphics processor or accelerator), and to the high-speed expansion ports 1250, which may accept various expansion cards (not shown). In some implementations, the low-speed controller 1260 is coupled to the storage device 1230 and a low-speed expansion port 1290. The low-speed expansion port 1290, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 1200 may be implemented in a number of different forms, as shown in the figure. For example, it may be implemented as a standard server 1200a or multiple times in a group of such servers 1200a, as a laptop computer 1200b, or as part of a rack server system 1200c.

It will be appreciated that additional signals and biometrics (e.g., other orthostatic metric values determined by the PPG evaluation unit) may be collected via patient monitor for use (e.g., comparison or assessment) in making a diagnosis of a disorder or disease (e.g., orthostatic intolerance), which are listed and described in TABLE 1 below. These metrics may be incorporated into one or more of the methods described herein, and may supplement or replace the metrics (e.g., OVH1, OHV2) described above.

TABLE 1

| | | |
|---|---|---|
| Symbol | Signal | Physiological Relevant |

Temporal signals: One measurement per sample.
Sampling frequency can vary between 10-200 Hz.

| | | |
|---|---|---|
| PPG, | Photoplethysmography signal. | PPG increases with blood volume. We |
| PPGi | Inverted raw sensor signal so an | monitor changes in PPG signal due to |
| | increase in PPG signal signifies | pulsation, and due to a postural change |
| | an increase in blood volume. | (supine to sit, supine to stand, sit to |
| | | stand, tilting on a medical table, etc.). |
| PPGb | PPG baseline signal. This is the | Signal formed by taking the first point |
| | envelope signal. | of each beat (beginning of systole), and |
| | | interpolating all other datapoints in |
| | | between. Could also be calculated by |
| | | using low pass or other filters. |
| PPG-br, | PPG signal with baseline | PPG-br represents changes in the blood |
| PVW | removed. Calculated as PPG − | volume only due to pulsation. It |
| | PPGb. | eliminates changes due to postural |
| | | change or drift. May be used to |
| | | visually observe changes in pulse |
| | | properties (contour, amplitude, volume, |
| | | etc.). |
| DIF | Difference signal. This represents | DIF indicates the rate of change at |
| | the derivative or slope of the PPG | which blood volume increases in the |
| | waveform, and is also referred to | earlobe. We anticipate it is related to |
| | as an edge detector kernel. One | rate of blood volume being ejected |
| | way to calculate it is DIF = | from the heart, and may be related to |
| | PPG[i] − PPG[i-1] | heart contractility. Can be used to |
| | | identify systole and diastole portions of |
| | | waveform (+DIF = systole, −DIF = |
| | | diastole). |
| Acc, | Accelerometer | Used to determine when an individual |
| AccX | | moved or changes positions during a |
| | | stand up test. A triple axis |
| | | accelerometer can provide not only |
| | | acceleration, but also tilt/orientation |
| | | measurement, which can be used to |
| | | identify when an individual stands. Can |
| | | also be used to avoid or reduce motion |
| | | artifacts. |
| BVI, | Blood volume index. Calculated | Signifies the relative change in blood |
| PPGn | as PPG/(the slow moving average | volume in small and intermediate |
| | of the PPG signal). | timescales. For example, if the BVI |
| | | dropped by 2% during a stand up test, |
| | | that indicates the the blood volume |
| | | change was. |
| | | Similar to how PPG Pulsation index |
| | | divides the AC portion by the DC |
| | | portion or the PPG signal (PI = |
| | | AC/DC). |

Beat features: features extracted from each pulsation (heart beat). One measurement per beat.

| | | |
|---|---|---|
| SB | Systole Baseline | First PPG value in a pulsation |
| PAMP, | Pulse amplitude | Maximum increase in blood volume |
| PA, PP | | during pulsation. Related to pulse |
| | | pressure, the difference in pressure |
| | | (systole - diastole) in the capillary bed. |
| PI | Pulsation index, equal to the | Pulsation index is often used in |
| | Pulse amplitude divided by the | Photoplethysmography to determine |
| | DC value of the sensor raw | the quality of the signal. If the PI is |
| | signal. Usually, the sensor raw | very low, either the sensor is not placed |
| | signal is somewhere | properly, or the blood perfusion in the |
| | | earlobe is low |
| DAMP, | Max slope of PPG signal during | Related to contractility of the heart. |
| DA | systole. Equal to the max of the | Contractility related to cardiac preload |
| | difference signal. | via the Frank-Starling rule |
| PV | Pulse volume | Related to stroke volume. Although we |
| | | are not measuring stroke volume |
| | | directly, the PV is related to the |
| | | volume of blood flowing through the |
| | | capillary bed during a pulsation. |
| | | Changes in PV are related to changes |
| | | in stroke volume (shown in data) |
| IBI | Interbeat interval | Time between this beat and adjacent |
| | | beat. Analogous to RR intervals in |
| | | ECG measurements. Changes in IBI |
| | | are related to cardioacceleration, and |
| | | orthostatic measurements |
| HR | Heart rate (beats per minute). | Used to determine heart rate changes |
| | Calculated from the IBI | during orthostasis |
| PAMPn, | PAMP normalized by PAMP in | % change in pulse amplitude after |
| PAn | the supine position | standing |
| DAMPn, | DAMP normalized by DAMP in | % change in contractility. after |
| DAn | the supine position | standing |
| PVn | PV normalized by PV in the | % change in pulse volume after |
| | supine position | standing |
| TR | Rise time. Time duration of the | Duration of the rise time is related to |
| | rising portion of the pulse PPG | hemodynamics of the heart and |
| | signal. | vasculature. Specifically, the TR is the |
| | | time duration when the capillary bed is |
| | | filling with blood, and may be related |
| | | to the duration of the systole in the |
| | | heart. |
| TF | Fall time. Time duration of the | Duration of the diastole is related to |
| | falling portion of the pulse | hemodynamics of the heart and |
| | | vasculature. Specifically, the TF is the |
| | | time duration when the capillary bed is |
| | | filling with blood, and may be related |
| | | to the duration of the systole in the |
| | | heart. |
| TRn | Normalized Rise time. Time | The percentage of time the blood |
| | duration of systole portion of the | volume is rising vs. falling can indicate |
| | pulse, divided by the total | cardiovascular parameters including |
| | duration of the pulse (interbeat | systole duration and drainage to the |
| | interval) | lower extremities. For example, when |
| | | standing, drainage happens faster due |
| | | to gravity. |
| TFn | Normalized Fall Time. Time | See above |
| | duration of diastole portion of the | |
| | pulse, divided by the total | |
| | duration of the pulse (interbeat | |
| | interval) | |
| SPO2, | Pulse oxygen level | Blood oxygen levels may decrease |
| Pox | | upon standing, due to decreased blood |
| | | flow and oxygen perfusion to the head. |
| | | This may induce lightheadedness and |
| | | other symptoms. |

Orthostatic Metrics: One measurement per postural transition.
Also referred to as Orthostatic Vital Signs (OVS).

| | | |
|---|---|---|
| OVH1 | Orthostatic hypovolemia 2. | Change in blood volume in the head |
| | Measured as decrease in PPG | area immediately after postural change. |
| | signal immediately after postural | |
| | change | |
| OVH2 | Orthostatic hypovolemia 2 | Change in blood volume in the head |
| | | area after postural homeostasis |
| | | (orthostasis) has been reached |
| | | (t = t0 + OTC) |
| OHV1n | Normalized OHV1 OHV1n = | Ratio of the maximal change in blood |
| | OHV1/Asupine | volume immediately upon standing vs. |
| | | the change in blood volume during a |
| | | single pulse in the supine position. This |
| | | is a way to normalize the OHV in a |
| | | way that may be less sensitive to sensor |
| | | placement and individual differences in |
| | | perfusion. |
| OHV2n | Normalized OHV2 OHV2n = | Ratio of the change in blood volume in |
| | OHV2/PAsupine | the head area after postural |
| | | homeostasis has been reached (t = t0 + |
| | | OTC). the change in blood volume |
| | | during a single pulse in the supine |
| | | position |
| OHV1p | Proportional OHV OHV1p = | % deficit in blood volume immediately |
| | OHV1/PPGavg, supine | after orthostasis (% volume loss |
| | | compared to supine)This is based on a |
| | | derivation PPG1 = 10 exp(k1) PPG2 = |
| | | 10 exp(k2) PPG1 PPG2 = exp(k1) |
| | | exp(k2) Δk = k11 − k21 = |
| | | ln(PPG2/PPG1) ~(PPG2/PPG1 − 1) = |
| | | PPG2 − PPG1 PPG1 = OHV PPG |
| | | supine |
| OHV2p | Percentage OHV2 | % deficit in blood volume after |
| | | orthostasis (% volume loss compared |
| | | to supine) |
| OTC | Orthostatic time constant | Time to complete orthostasis, ie time to |
| | | achieve homeostasis (equilibrium) in |
| | | standing position. Individuals with |
| | | neurological or cardiovascular |
| | | impairment may have longer OTC than |
| | | individuals with a healthy autonomic |
| | | response. Some individuals may never |
| | | stabilize in the standing position, in |
| | | which case OTC is undefined. |
| POT | Postural orthostatic tachycardia | Maximum increase in heart rate during |
| | | orthostasis |
| PVR, | Maximum change in pulse | % decrease in pulse volume |
| OPVR, | volume immediately after | immediately after postural transition. |
| OPV1, | standing, divided by pulse | May be related to reduction in stroke |
| PV1 | volume in the supine position. | volume or ejection fraction. |
| PAR, | Maximum change in pulse | % decrease in pulse amplitude |
| PA1 | amplitude immediately after | immediately after postural transition. |
| | standing, divided by pulse | May be related to reduction in blood |
| | amplitude in supine position. | pressure or stroke volume. |
| DAR, | Maximum change in difference | % decrease in difference amplitude |
| DA1 | amplitude immediately after | immediately after postural transition. |
| | standing, divided by difference | May be related to reduction in |
| | amplitude in the supine position | contractility or blood pressure. |
| PV2 | Pulse volume reduction after | % decrease in pulse volume after |
| | orthostasis has completed (t = t0 + | orthostasis. May indicate sustained |
| | OTC), divided by pulse volume | orthostatic hypotension, reduced |
| | in supine position | vasoconstriction, reduced preload |
| PA2 | Pulse amplitude reduction after | % decrease in pulse amplitude after |
| | orthostasis has completed (t = t0 + | orthostasis. May indicate sustained |
| | OTC), divided by pulse | orthostatic hypotension, reduced |
| | amplitude in supine position | vasoconstriction, or reduced preload. |
| DA2 | Differential amplitude reduction | % decrease in difference amplitude |
| | after orthostasis has completed (t = | after orthostasis. May indicate |
| | t0 + OTC), divided by | sustained orthostatic hypotension, |
| | difference amplitude in supine | reduced vasoconstriction, or reduced |
| | position | preload. |
| Pox1 | Initial reduction in SPO2 | Decrease in blood oxygen in the head |
| | immediately after standing (% | area initially upon standing. This can |
| | SPO2) | be due to decreased loss of blood |
| | | volume upon standing |
| Pox2 | Reduction in SPO2 after | Decrease in blood oxygen in the head |
| | orthostasis has been completed | area after orthostasis. Sustained |
| | (% SPO2) | decrease in oxygen may cause an |
| | | individuals to feel brain fog or fatigue |
| | | when standing for some time |

It will be appreciated that Artificial Intelligence (AI) may be incorporated to one or more of the methods/processes (including individual steps) discussed herein, including but not limited to continuous monitoring and assessment of the orthostatic metrics measured by patient monitor and evaluation of the metrics for diagnosis. The computer devices and/or servers described herein may us machine learning algorithms or deep learning techniques to process the sensed data (e.g., detect the PPG waveform including one or more of the orthostatic metrics described herein), make comparisons against normal values, and provided assessments based on the comparisons.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations have been described. Nevertheless, it will be understood that various modifications may be made without departing from the spirit and scope of the disclosure. Accordingly, other implementations are within the scope of the following claims.

What is claimed is:

1. A method comprising:
during a stand-up or sit-up evaluation:
generating, by data processing hardware, photoplethysmogram (PPG) data and motion data continuously;
in response to detecting, based on the motion data, that an angle associated with a user's posture indicates that the user is in a supine position, associating, by the data processing hardware, the PPG data with a first phase;
in response to detecting, based on the motion data, that the angle associated with the user's posture indicates that the user is in a standing up position or a sitting up position, associating, by the data processing hardware, the PPG data with a third phase; and
determining one or more orthostatic metrics based on the PPG data associated with the first phase and the PPG data associated with the third phase.

2. The method of claim 1, further comprising:
in response to detecting, based on the motion data, that the angle associated with the user's posture indicates that the user is in a process of standing up or a sitting up, associating, by the data processing hardware, the PPG data with a second phase.

3. The method of claim 1, further comprising, providing, by the processing hardware, an indication or prompt for when to stand up or sit up during the first stand-up or sit-up evaluation.

4. The method of claim 1, further comprising, informing, by the processing hardware, the user to complete transition to the stand-up position or sit-up position within a predetermined time and to remain stationary thereafter.

5. The method of claim 1, further comprising, informing, by the processing hardware, the user to complete transition to the stand-up position or sit-up position within a predetermined time and to remain stationary thereafter.

6. The method of claim 1, further comprising, providing, by the processing hardware, the one or more orthostatic metrics to the user or healthcare provider associated with the user.

7. The method of claim 1, further comprising:
in response to detecting, based on the motion data, that the angle associated with the user's posture indicates that the user is in a process of standing up or a sitting up, associating, by the data processing hardware, the PPG data with a third phase.

8. The method of claim 1, wherein:
determining the one or more orthostatic metrics includes determining orthostatic time constant (OTC) including:
determining an ending time the first phase;
determining a starting time within the third phase when the PPG data become stabilized; and
determining the OTC based on a time interval between the ending time the first phase and the starting time the PPG data become stabilized.

9. The method of claim 1, wherein:
determining the one or more orthostatic metrics includes determining orthostatic pulse volume reduction (OPVR) including:
generating a pulse waveform based on the PPG data;
generating a pulse volume waveform based on the pulse waveform;
generating a normalized pulse volume waveform based on the pulse volume waveform; and
determining the OPVR based on a difference between a maximum value in the normalized pulse volume waveform associated with first phase and a minimum value in the normalized pulse volume waveform associated with the third phase.

10. The method of claim 1, wherein:
determining the one or more orthostatic metrics includes determining orthostatic postural orthostatic tachycardia pulse volume reduction (POT) including:
generating a heart rate waveform based on the PPG data;
determining a minimum value in the heart rate waveform associated with the first phase and a maximum value in the heart rate waveform associated with the third phase;
determining the POT based on a difference between the minimum value and the maximum value.

11. The method of claim 1, wherein:
determining the one or more orthostatic metrics includes determining a first orthostatic hypovolemia (OHV1) including:
determining a minimum value in the PPG data associated with the first phase;
determining a minimum value in the PPG data associated with the third phase; and
determining the OHV1 based on a difference between the minimum value in the PPG data associated with the first phase and the maximum value in the PPG data associated with the third phase.

12. The method of claim 1, wherein:
determining the one or more orthostatic metrics includes determining a second orthostatic hypovolemia (OHV2) including:
determining a minimum value in the PPG data associated with the first phase;
determining a starting time within the third phase when the PPG data become stabilized;
determining a minimum value in the PPG associated with the third phase time after the PPG data becomes stabilized; and
determining OHV2 based on a difference between the minimum value in the PPG data associated with the first phase and the minimum value in the PPG associated with the third phase time after the PPG data becomes stabilized.

13. The method of claim 1, further comprising:
determining a likelihood of the user having an orthostatic disorder based on the one or more orthostatic metrics.

14. The method of claim 1, wherein the PPG data is normalized based on a temperature.

15. A device comprising:
one or more processors; and
memory storing instructions that, when executed by the one or more processors, cause the monitoring device to:
obtain photoplethysmogram (PPG) data and motion data;
in response to determination, based on the motion data, that an angle associated with a user's posture indicates that the user is in a supine position, associate the PPG data with a first phase;
in response to determination, based on the motion data, that the angle associated with the user's posture indicates that the user is in a standing up position or a sitting up position, associate the PPG data with a third phase; and
determine one or more orthostatic metrics based on the PPG data associated with the first phase and the PPG data associated with the third phase.

16. The device of claim 15, wherein the instructions further cause the device to associating the PPG data with a second phase in response to determination, based on the motion data, that the angle associated with the user's posture indicates that the user is in a process of standing up or a sitting up, associating, by the data processing hardware, the PPG data with a second phase.

17. The device of claim 15, wherein the instructions further cause the device to provide an indication or prompt for when to stand up or sit up during the first stand-up or sit-up evaluation.

18. The device of claim 15, wherein the instructions further cause the device to inform the user to complete transition to the stand-up position or sit-up position within a predetermined time and to remain stationary thereafter.

19. The device of claim 15, wherein the instructions further cause the device to informing the user to complete transition to the stand-up position or sit-up position within a predetermined time and to remain stationary thereafter.

20. The device of claim 15, wherein the instructions further cause the device to providing the one or more orthostatic metrics to the user or healthcare provider associated with the user.

21. The device of claim 15, wherein the instructions further cause the device to associate the PPG data with a third phase, in response to determination, based on the motion data, that the angle associated with the user's posture indicates that the user is in a process of standing up or a sitting up.

22. The device of claim 15, wherein the instructions further cause the device to determine a likelihood of the user having an orthostatic disorder based on the one or more orthostatic metrics.

23. A non-transitory computer-readable medium comprising instructions that, when executed by one or more processors of a device, cause the deice to:
generate photoplethysmogram (PPG) data and motion data continuously;
in response to determination, based on the motion data, that an angle associated with a user's posture indicates that the user is in a supine position, associate the PPG data with a first phase;
in response to determination, based on the motion data, that the angle associated with the user's posture indicates that the user is in a standing up position or a sitting up position, associate the PPG data with a third phase; and
determine one or more orthostatic metrics based on the PPG data associated with the first phase and the PPG data associated with the third phase.

24. A system comprising:
a monitoring device comprising:
one or more first processors; and
first memory storing first instructions that, when executed by the one or more first processors, cause the monitoring device to:
generate photoplethysmogram (PPG) data and motion data continuously;
in response to determination, based on the motion data, that an angle associated with a user's posture indicates that the user is in a supine position, associate the PPG data with a first phase;
in response to determination, based on the motion data, that the angle associated with the user's posture indicates that the user is in a standing up position or a sitting up position, associate the PPG data with a third phase;
determine one or more orthostatic metrics based on the PPG data associated with the first phase and the PPG data associated with the third phase; and
providing the one or more orthostatic metrics to a computing device; and
the computing device comprising:
one or more second processors; and
second memory storing first instructions that, when executed by the one or more second processors, cause the computing device to:
store the one or more orthostatic metrics form the monitoring device.
